(19) United States
(12) Patent Application Publication
(10) Pub. No.: US 2024/0366545 A1
(43) Pub. Date: Nov. 7, 2024

(54) ANTIBACTERIAL PORPHYRIN NANOPARTICLES AND METHODS FOR MAKING AND USING THE SAME

(71) Applicant: University of Georgia Research Foundation, Inc., Athens, GA (US)

(72) Inventors: Hitesh Handa, Athens, GA (US); Anil Kumar, Winder, GA (US); Elizabeth J. Brisbois, Athens, GA (US)

(21) Appl. No.: 18/682,184

(22) PCT Filed: Aug. 9, 2022
(86) PCT No.: PCT/US2022/074684

§ 371 Date: Feb. 8, 2024

Related U.S. Application Data
(60) Provisional application No. 63/231,623, filed on Aug. 10, 2021.

(51) Int. Cl. A61K 31/444; A01N 25/12; A01N 43/40; A01P 1/00; A61K 9/51; A61L 17/00; A61L 29/16; A61P 31/04

(52) U.S. Cl. CPC A61K 31/444; A01N 25/12; A01N 43/40; A01P 1/00; A61K 9/5123; A61K 9/5169; A61L 17/005; A61L 29/16; A61P 31/04; A61L 2300/204; A61L 2300/224; A61L 2300/404; A61L 2400/12

(57) ABSTRACT

Described herein are antibacterial nanoparticles composed of a porphyrin and a nitric oxide donor. In one aspect, the antibacterial nanoparticles are produced by the method comprising (a) admixing a porphyrin with glutathione or a pharmaceutically acceptable salt or ester thereof to produce a first compound and (b) reacting the first compound with a nitric oxide compound, wherein the nitric oxide compound forms a covalent bond with glutathione or the pharmaceutically acceptable salt or ester. The antibacterial nanoparticles are dynamic in their ability to specifically target pathogenic infections while remaining nontoxic towards mammalian cells and can also be used as medical device coatings to prevent infections as well as in treatment and management of diseases like cancer and autoimmune skin disorders.

### CROSS-REFERENCE TO RELATED APPLICATIONS

This application claims the benefit of and priority to co-pending U.S. Provisional Patent Application No. 63/231,623, filed on Aug. 10, 2021, the contents of which are incorporated by reference herein in their entireties.

### STATEMENT REGARDING FEDERALLY SPONSORED RESEARCH OR DEVELOPMENT

This invention was made with government support under grant number RO1 HL134899, awarded by the National Institutes of Health. The government has certain rights in the invention.

### BACKGROUND

Recent developments in light-controlled/light-induced therapy (e.g., photodynamic and photothermal therapies) are promising strategies to prevent and suppress bacterial infections, which remain one of the most critical issues in the healthcare sector. Every year, approximately 10 million people die from infection-related complications thereby constituting a threat to patient mortality rates in the world.¹ Despite the development of many antibacterial compounds and agents, many strains of bacteria have developed unprecedented multi-drug resistance (MDR), which is a global public health challenge.^{2, 3} Poor drug solubility, pharmacokinetics, toxicity, and membrane transport limitations affect the potency of many antibiotics and limit their ability to achieve their maximum therapeutic potential.^{2, 4} Nanotechnology is an emerging technology with several distinct characteristics, which make it a unique and favorable platform to prevent infection by suppressing pathogens.^{5, 6} Nanomaterials have demonstrated their potential in improving drug solubility, stability, and biocompatibility,⁷ and also can be exploited for targeted drug delivery (organ- or region-specific),^{8, 9} and controlled drug release in response to stimuli such as pH^{10, 11} temperature, and light.¹² In addition to pharmaceutical applications, nanomaterials are also now being investigated for use in new medical devices and has opened up newer unexplored vistas in the field of medical devices.¹³

Antimicrobial photodynamic therapy (APDT) has drawn increasing attention for its ability to kill multi-drug resistance (MDR) pathogenic bacteria coupled with its low tendency to induce drug resistance.¹⁴ This provides significant advantages over existing antimicrobial therapies.^{14, 15} APDT is equally effective at eradicating both MDR strains as well as native bacterial strains because the light-induced therapy is localized to light-irradiated regions. Secondly, the effect of APDT on microorganisms is much more rapid compared to other antimicrobial agents, and there is no evidence of APDT resistance to-date.¹⁴ APDT uses a non-toxic compound to cause microbial cell death through a process called lethal photosensitization.¹⁶ Therefore, APDT can potentially be used as a powerful tool to elicit bactericidal activity via PS molecules.¹⁷ The PS molecules become activated by specific wavelengths of light in the visible (400-700 nm), ultraviolet (200-400 nm), and/or near infrared (700-1500 nm) spectral regions.¹⁷ These nontoxic PS molecules have the ability to release reactive oxygen species (ROS) such as singlet oxygen (¹O₂), peroxide (O₂), superoxide (O₂·), and hydroxyl radicals (·HO) when irradiated with light, killing target bacterial cells more efficiently.¹⁸ The abundance of these ROS results in oxidative damage to microorganisms through disruption of membrane proteins, lipids, and genetic materials (DNA/RNA), or irreversible alterations of their metabolic activities inducing cell death.^{19, 20}

Currently, most NP based PS are derived from metal and metal-based nanomaterials (gold and silver nanoparticles, zinc oxide, copper nanoparticles, etc.) which act as a PS due to their surface plasmonic behavior and therapeutic potential in biological systems.²¹. However, a major limitation to PDT in such cases is the lack of effective selectivity, water solubility, and metal ion toxicity.^{22, 23} Additionally, due to their intrinsic physiochemical properties, these NPs show potential toxicity towards cellular systems and can cause cellular damage.^{24, 25} Therefore, various NPs encounter difficulty towards clinical translation, and a large number of such metal-based NPs remain largely unapproved by the Food and Drug Administration (FDA) for medical use.^{25, 26} These NPs are synthesized using surfactants like CTAB to ensure monodispersity and stability among the NPs which may also contribute towards cellular toxicity.²⁷ CTAB plays an important role in controlling the size and shape of the metal NPs, making it imperative in the synthesis of most gold²⁸ and silver²⁷ nanoparticles. In a recent study, silver, zinc oxide, copper, and other composite materials developed using CTAB as a stabilizing agent were found to be toxic to mammalian cells and caused significant reduction in cell viability.²⁹ CTAB is toxic at concentrations as low as 10 μM because the positively charged CTAB is highly attracted to the negatively charged cell membrane, which causes the breakdown of the cell membrane and leads to cytotoxicity towards normal mammalian cells.³⁰

Recently porphyrins have been investigated as potential PSs in APDT due to their unique physiochemical and biochemical properties.³¹ These molecules show very intense absorption bands in the visible region and high singlet oxygen quantum yield due to their large TT-conjugated aromatic domains.³² The highly stable light-absorbing nature of porphyrins and their ability to release ROS in the presence of oxygen makes them ideal candidates for use in APDT.^{33, 34} The amphiphilic nature of the porphyrins bearing one or more positive charges to interact with cell surfaces makes them effective tools in PDT against microbial infections (ADPT) and cancers and has been recently investigated against autoimmune skin disorders.^{33, 35} However, most of the POP-NPs investigated used in ADPT as well as in cancer therapy are synthesized using different surfactants such as CTAB, MTAB, sodium dodecyl sulfate (SDS), sodium lauryl sulfate (SLS), and triblock copolymer (Poly(ethylene glycol)-block, Poly(propylene glycol)-block, Poly(ethylene glycol)) also known as P123.³⁶ These molecules show severe toxicity to normal mammalian cells, tissues and organs.³⁷

### SUMMARY

Described herein are antibacterial nanoparticles composed of a porphyrin and a nitric oxide donor. In one aspect, the antibacterial nanoparticles are produced by the method comprising (a) admixing a porphyrin with glutathione or a pharmaceutically acceptable salt or ester thereof to produce a first compound and (b) reacting the first compound with a nitric oxide compound, wherein the nitric oxide compound forms a covalent bond with glutathione or the pharmaceutically acceptable salt or ester. Under visible light irradiation the antibacterial nanoparticles generate high yields of singlet oxygen (¹O₂), hydroxyl radical (·HO), superoxide radical (O₂·), and peroxynitrite (ONOO⁻) free radicals that can enhance antimicrobial photodynamic therapy (APDT). The antibacterial nanoparticles are dynamic in their ability to specifically target pathogenic infections while remaining nontoxic towards mammalian cells and can also be used as medical device coatings to prevent infections as well as in treatment and management of diseases like cancer and autoimmune skin disorders.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 shows a schematic representation of GSH Stabilization (step-I) and Cys-NO functionalization (step-II) of POP-NPs.

FIGS. 2A-2F show the characterization of glutathione (GSH) stabilized, S-nitrosocysteine (Cys-NO) functionalized POP (GSH)-Cys-NO-NPs. (a) A representative SEM image of the octahedral POP (GSH)-Cys-NO-NPs (b) Corresponding TEM image of POP (GSH)-Cys-NO-NPs in water. (c) Dynamic light scattering analysis (Size measurements) of POP (GSH)-Cys-NO-NPs in water. The histogram shows that the average size of the NPs analyzed was 163±2.31 nm with an average edge length of 103±2.4 nm (d) Zeta-potential of POP (GSH)-Cys-NO-NPs show-32.13 mV in water. (e) UV-visible spectra of POP-NPs in water. The image (A, B, C) on the top right-hand corner shows the solution of POP-NPs in water. (f) Fluorescence spectra of the synthesized POP (GSH)-Cys-NO-NPs at different concentrations in water.

FIG. 3 shows the stabilization of POP-NPs with GSH was confirmed via FTIR with characteristic peaks for GSH in POP-GSH-NPs at 3315, 2535, and 1840 cm⁻¹ corresponding to N—H stretching, S—H stretching, and C═O stretching vibrations.

FIGS. 4A-4D shows the characterization of cetyl trimethylammonium bromide (CTAB) stabilized, porphyrin NPs (POP-CTAB-NPs). (a) A solution of 5,10,15,20-Tetra(4-pyridyl)-21H,23H-porphine in water containing 0.1 M HCL (1), POP-CTAB-NPs in water after synthesis (2). (b) A representative TEM image of the octahedral POP-CTAB-NPs in water (Scale bar 100 nm). (c) UV-visible spectra of POP-CTAB-NPs in water. (d) Reference spectra for POP-CTAB-NPs confirm the successful incorporation of CTAB in the NPs with asymmetrical C—H and symmetrical C—H stretching at 2960 and 2860 cm⁻¹, respectively.

FIGS. 5A-5B show (a) average NO release measurements (×10⁻¹⁰ mol NO mg⁻¹ min⁻¹) of POP (GSH)-Cys-NO-NPs (a,b) over 72 h at 37° C. with and without light exposure (*P<0.05). (b) NO release profiles of freshly prepared nanoparticles and nanoparticles stored at −20° C. for 30 days. No significant difference in the cumulative NO release was found before (black) or after (grey) 30 days of storage at −20° C., indicating that the NPs are a durable, tunable system for NO delivery.

FIGS. 6A-6E show the stability of POP-NPs in different solutions and storage conditions: (a) Samples of POP-NPs (mg/ml) in water, (a) 1× phosphate buffer (mg/ml) (c) cell culture medium (with 10% FBS) (d) complete Luria-Bertani (LB) bacteria culture medium. All the samples were kept for 2 h at room temperature and samples were imaged. (e, f) Storage stability of POP-NPs (10 mg/ml) in Milli-Q water at 4° C.

FIGS. 7A-7D show measuring reactive oxygen and nitrogen species with fluorescent probes. Figure shows the changes in fluorescence intensity produced by generation of (a) singlet oxygen (SOSG), (b), hydroxyl (HPF), (c) peroxynitrite (ONOO⁻) and, (d) superoxide radicals (DHE) in the presence of white light irradiation (dose, 46 J/cm²) with POP (GHS)-Cys-NO-NPs. The data is presented as mean±standard based on the result (N=6). The t-test has been used for the statistical significance determination. (*P<0.05; **P<0.01; ***P<0.001; ns, no significant difference).

FIGS. 8A-8C show the cytotoxicity evaluation of POP-NPs in fibroblast cells (3T3). Cells were exposed to different nanoparticles concentrations ranging from 2 to 500 μg/ml of POP-NPs (a) in dark and (b) light conditions. Viability of cells was determined using CCK-8 assay after 72 h. (c) Fluorescence microscopy of cell monolayers treated with POP (GSH)-Cys-NO-NPs and POP (CTAB)-NPs. Fibroblast cells were treated with 100 μg/ml of POP-NPs or left untreated as a control. After 72 h, the cells were stained with calcein-AM (green) and EthD-III (red) and observed under confocal laser scanning microscope. Scale bars, 50 μm. (*P<0.05; **P<0.01; ***P<0.001; ns, no significant difference).

FIG. 9 shows the brightfield microscopy images of mouse fibroblasts treated for 72 h with varied concentrations of POP-GSH/Cys-NO-NPs. Scale bars correspond to 200 μm.

FIGS. 10A-10B show (a) median fluorescence intensity (MFI) of Calcein-M, based on imaging results from the green channel Image4 and (b) MFI of Eth-Ill based on imaging results from the red channel in Image4. *P<0.05; **P<0.01; ***P<0.001; ns, no significant difference.

FIG. 11 shows the brightfield microscopy images of mouse fibroblasts treated for 72 h with varied concentrations of POP-CTAB-NPs. Scale bars correspond to 200 μm.

FIGS. 12A-12D show the qualitative and quantitative analysis of the effect of NO generation in living cells. (a) Confocal laser scanning microscopy imaging results. Mouse fibroblast cells incubated with 100 g/ml of POP-NPs for 6 h and irradiated with light (46 J/cm²) and further incubated for 24 h. The cells were stained using a fluorescent probe (DAM-FM) for NO detection and EthD-III (red, for cells with breached membrane) dye as a marker for DNA damage. Results show that the POP (GSH)-Cys-NO)-NPs were internalized by cells without causing noticeable cytotoxicity. POP (CTAB)-NPs resulted in a dramatic, observable alteration in cell morphology and toxicity towards the cells. (b) Median fluorescence intensity (MFI) of DAF-FM, based on imaging results from (a). (c) Relative fluorescence units (RFU). (d) MFI of EthD-III based on imaging results from the red channel in (a). Cells received the same treatment as for other radical measurement and the fluorescence activity was measured on a microreader instead of under a confocal microscope. *P<0.05; **P<0.01; ***P<0.001; ns, no significant difference.

FIGS. 13A-13D show the antibacterial effect of POP (GSH)-NPs and POP (GSH)-Cys-NO-NPs on (a, b) E. coli and (c, d) MRSA in the presence and absence of light. *P<0.05; **P<0.01; ***P<0.001; ns, no significant difference

FIGS. 14A-14D show the respiratory activity of (a, b) E. coli and (c, d) MRSA after treating with POP-GSH-NPs and POP-GSNO-NPs in the presence and absence of light. *P<0.05; **P<0.01; ***P<0.001; ns, no significant difference

FIGS. 15A-15E show the SEM images of E. coli (a,b) and MRSA (c,d). (a,c) Prior to treatment bacteria cells exhibited smooth intact surfaces with well-defined morphologies and sizes. (b,d) After treatment with NPs (red arrows) and light irradiation, significant damages to cell wall structure was observed (blue arrows). Scalebar=1 μm length. (d) Schematic showing the effect of photodynamic behavior of NPs on bacterial cell function.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions and Abbreviations

In describing and claiming the disclosed subject matter, the following terminology will be used in accordance with the definitions set forth below.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an antibacterial nanoparticle” includes, but are not limited to, mixtures or combinations of two or more such antibacterial nanoparticles (e.g., a plurality of antibacterial nanoparticles), and the like.

It should be noted that ratios, concentrations, amounts, rates, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance and instances where it does not.

As used herein, the term “biocompatible,” with respect to a substance or fluid described herein, indicates that the substance or fluid does not adversely affect the short-term viability or long-term proliferation of a target biological particle within a particular time range.

The terms “antimicrobial” and “antimicrobial characteristic” refer to the ability to kill and/or inhibit the growth of microorganisms. A substance having an antimicrobial characteristic may be harmful to microorganisms (e.g., bacteria, fungi, protozoans, algae, and the like). A substance having an antimicrobial characteristic can kill the microorganism and/or prevent or substantially prevent the growth or reproduction of the microorganism.

The term “antimicrobial effective amount” as used herein refers to that amount of the compound being administered which will kill microorganisms or inhibit growth and/or reproduction thereof to some extent (e.g. from about 5% to about 100%). In reference to the compositions or articles of the disclosure, an antimicrobial effective amount refers to that amount which has the effect of diminishment of the presence of existing microorganisms, stabilization (e.g., not increasing) of the number of microorganisms present, preventing the presence of additional microorganisms, delaying or slowing of the reproduction of microorganisms, and combinations thereof.

The terms “bacteria” or “bacterium” include, but are not limited to, gram positive and gram negative bacteria. Bacteria can include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anabaena affinis and other cyanobacteria (including the Anabaena, Anabaenopsis, Aphanizomenon, Camesiphon, Cylindrospermopsis, Gloeobacter Hapalosiphon, Lyngbya, Microcystis, Nodularia, Nostoc, Phormidium, Planktothrix, Pseudoanabaena, Schizothrix, Spirulina, Trichodesmium, and Umezakia genera) Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Propionibacterium, Proteus, Porphyrimonas, Prevotella, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof. The gram-positive bacteria may include, but is not limited to, gram positive Cocci (e.g., Streptococcus, Staphylococcus, and Enterococcus). The gram-negative bacteria may include, but is not limited to, gram negative rods (e.g., Bacteroidaceae, Enterobacteriaceae, Vibrionaceae, Pasteurellae and Pseudomonadaceae).

The term “antimicrobial effective amount” as used herein refers to that amount of the compound being administered/released that will kill microorganisms or inhibit growth and/or reproduction thereof to some extent (e.g. from about 5% to about 100%). In reference to the compositions or articles of the disclosure, an antimicrobial effective amount refers to that amount which has the effect of diminishment of the presence of existing microorganisms, stabilization (e.g., not increasing) of the number of microorganisms present, preventing the presence of additional microorganisms, delaying or slowing of the reproduction of microorganisms, and combinations thereof. Similarly, the term “antibacterial effective amount” refers to that amount of a compound being administered/released that will kill bacterial organisms or inhibit growth and/or reproduction thereof to some extent (e.g., from about 5% to about 100%). In reference to the compositions or articles of the disclosure, an antibacterial effective amount refers to that amount which has the effect of diminishment of the presence of existing bacteria, stabilization (e.g., not increasing) of the number of bacteria present, preventing the presence of additional bacteria, delaying or slowing of the reproduction of bacteria, and combinations thereof.

As used herein, the term “subject” includes humans, mammals (e.g., cats, dogs, horses, etc.), birds, and the like. Typical subjects to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications. In some embodiments, a system includes a sample and a host. The term “living host” refers to the entire host or organism and not just a part excised (e.g., a liver or other organ) from the living host.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, substantially preventing spread of disease, amelioration or palliation of the disease state, and remission (partial or total) whether detectable or undetectable.

The term “visible light” is the part of the electromagnetic spectrum that is visible to the human eye, which is in the range of about 380 to about 750 nanometers.

The term “substituted” refers to any one or more hydrogens on the designated atom that can be replaced with a selection from the indicated group, provided that the designated atom's normal valence is not exceeded.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

The alkyl group can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

“Aryl”, as used herein, refers to C5-C10-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O) A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas-S(O) A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a shorthand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O) A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

### Discussion

In accordance with the purpose(s) of the present disclosure, described herein are antibacterial nanoparticles composed of a porphyrin and a nitric oxide donor. The use of glutathione or the pharmaceutically acceptable salt or ester thereof as a stabilizer for the synthesis of the antibacterial nanoparticles imparts unique biological properties not recognized or appreciated in the current state of the art.

In one aspect, the antibacterial nanoparticles are produced by the method comprising (a) admixing a porphyrin with glutathione or a pharmaceutically acceptable salt or ester thereof to produce a first compound and (b) reacting the first compound with a nitric oxide compound, wherein the nitric oxide compound forms a covalent bond with glutathione or the pharmaceutically acceptable salt or ester.

In one aspect, the porphyrin used to produce the antibacterial nanoparticles described herein has the structure I

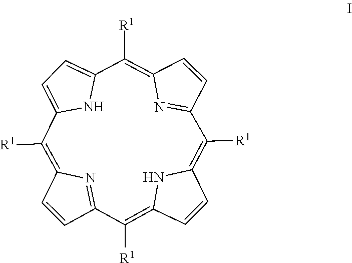

wherein R¹ are each a substituted or unsubstituted aryl group, a substituted or unsubstituted alkyl group, a substituted or unsubstituted cycloalkyl group, a carboxylic acid or ester, an amino group, or an azido group.

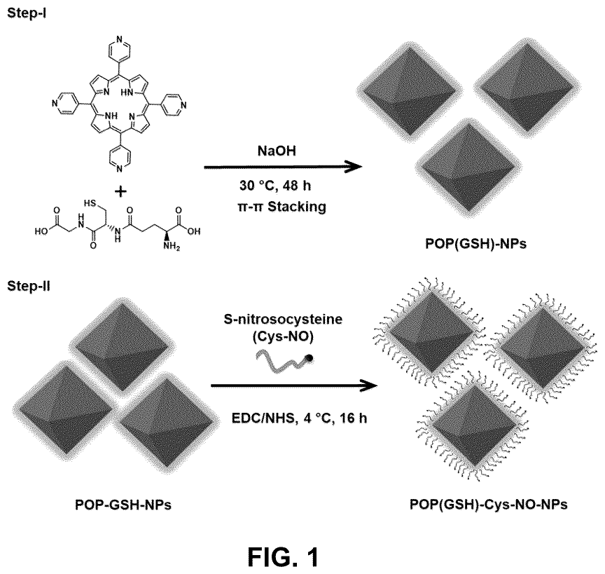

The selection of R¹ in structure I can vary depending upon the desired properties of the antibacterial nanoparticles. For example, R¹ can be selected to modify the hydrophilicity, hydrophobicity, and aliphaticity of the porphyrin, which can influence nanoparticle assembly, size, morphology, and biological activity. In one aspect, R¹ is a heteroaryl group. In another aspect, R¹ is a pyridinyl group. In another aspect, the porphyrin is 5, 10, 15,20-tetra(4-pyridyl)-21H,23H-porphine, the structure of which is provided in FIG. 1.

In another aspect, the porphyrin further includes a transition metal coordinated to the porphyrin. Not wishing to be bound by theory, transition metal ions can perform as a catalyst towards S-nitrosothiol decomposition, which in turn can modify or tune the release kinetics of nitric oxide from the antibacterial nanoparticles. In one aspect, the transition metal ions can coordinate to porphyrin of structure by through the nitrogen atom of the pyrrole rings. Examples of transition metals useful herein include, but are not limited to, copper, zinc, manganese, cobalt, or cadmium.

The porphyrin and glutathione or the pharmaceutically acceptable salt or ester thereof are admixed with one another in a solvent. In one aspect, the solvent is water, an organic solvent, or a combination thereof. In one aspect, an aqueous solution of the porphyrin with an acid is added to an aqueous solution of glutathione or the pharmaceutically acceptable salt or ester thereof and a base. In one aspect, the acid in the porphyrin solution is a strong acid such as, for example, hydrochloric acid, phosphoric acid, or sulfuric acid. The concentration of the acid can vary and is in the range of from about 0.1 M to 1.0 M. In one aspect, the base present in the solution composed of glutathione or the pharmaceutically acceptable salt or ester thereof is an alkali metal hydroxide or alkaline earth metal hydroxide. In one aspect, the base is sodium hydroxide. The concentration of the base can vary and is in the range of from about 0.001 M to 0.1 M.

The relative amount of porphyrin to glutathione or the pharmaceutically acceptable salt or ester thereof can vary. In one aspect, the molar ratio of porphyrin to glutathione or the pharmaceutically acceptable salt or ester thereof is from about 0.5:1 to about 2:1, or about 0.5:1, 0.75:1, 1:1, 0.25:1, 1.5:1, 1.75:1, or 2:1, where any value can be a lower and upper endpoint of a range (e.g., 0.75:1 to 1.25:1).

In certain aspects, the reaction product between the porphyrin and glutathione or the pharmaceutically acceptable salt or ester thereof, which is referred to herein as the first product, can be isolated and purified. Non-limiting techniques for isolating and purifying the reaction product can be found in the Examples. Not wishing to be bound by theory, the porphyrin and glutathione or the pharmaceutically acceptable salt or ester thereof undergo a self-assembly process. Fourier transform infrared spectroscopy indicates the thiol group of glutathione or the pharmaceutically acceptable salt or ester thereof (2535 cm⁻¹) is present.

The reaction product between the porphyrin and glutathione or the pharmaceutically acceptable salt or ester thereof is subsequently reacted with a nitric oxide compound. In one aspect, the nitric oxide compound is an S-nitroso thiol of formula O═N—S—R, where R can be an alkyl or aryl moiety. Reference to alkyl and aryl moieties includes substituted and unsubstituted alkyl and aryl moieties, respectively. In an aspect, the alkyl, substituted alkyl, aryl, or substituted aryl moiety can comprise from about 5 to about 20 carbons.

In one aspect, the nitric oxide compound is a compound that possesses reactive groups such as amino groups and carboxyl groups that can react with glutathione or the pharmaceutically acceptable salt or ester thereof to produce new amide bonds. In one aspect, the nitric oxide compounds can be an amino acid moiety with a thiol group. In another embodiment, the nitric oxide compound is S-nitroso-N-acetyl-penicillamine, S-nitroso-N-acetylcysteine, S-nitroso-N-acetyl cysteamine, S-nitrosoglutathione, S-nitrosocysteamine-glutathione, methyl S-nitrosothioglycolate, nitrosated cysteine, or any combination thereof.

The first product as described above and the nitric oxide compound are admixed with one another in a solvent. In one aspect, the solvent is water, an organic solvent, or a combination thereof. In one aspect, in order to facilitate the reaction between first product as described above and the nitric oxide compound, dimethylaminopropyl)-N-ethylcarbodiimide hydrochloride (EDC) and N-hydroxysuccinimide (NHS) can be used to produce the antibacterial nanoparticles described herein. Non-limiting techniques for isolating and purifying the antibacterial nanoparticles can be found in the Examples.

The nitric oxide compound forms a covalent bond with glutathione or the pharmaceutically acceptable salt or ester thereof. Not wishing to be bound by theory, the nitric oxide compound reacts with glutathione to form a new amide bond. The thiol group of glutathione is also capable of forming a covalent bond with the nitric oxide compound.

The dimensions and properties of the antibacterial nanoparticles can be characterized by a number of different techniques. In one aspect, scanning electron microscopy (SEM) and transmission electron microscopy (TEM) can be used to evaluate the geometric dimensions of the antibacterial nanoparticles. In one aspect, the antibacterial nanoparticles described herein have a well-defined external morphology with an average edge length of about 140 nm to about 150 nm, octahedral geometry, and uniform size distribution as determined by scanning electron microscopy (SEM) shows that. In another aspect, the antibacterial nanoparticles have a clear edge length of about 100 nm to about 120 nm in length and a well-defined 3D-octahedral shape, uniform dispersion, and show uniform electron contrast without apparent defects as determined by transmission electron microscopy (TEM).

In one aspect, the antibacterial nanoparticles described herein have an average size of about 100 nm to about 200 nm, or about 100 nm, 110 nm, 120 nm, 130 nm, 140 nm, 150 nm, 160 nm, 170 nm, 180 nm, 190 nm, or 200 nm, where any value can be a lower and upper endpoint of a range (e.g., 150 nm to 170 nm) as determined by dynamic light scattering (DLS). In another aspect, antibacterial nanoparticles have a polydispersity index from about 0.1 to about 0.3, or about 0.1, 0.15, 0.2, 0.25, or 0.3, where any value can be a lower and upper endpoint of a range (e.g., 0.1 to 0.2). The antibacterial nanoparticles described herein have a narrow size distribution.

The antibacterial nanoparticles described herein possess a negatively charged surface. In one aspect, the antibacterial nanoparticles have a zeta potential of from about-20 mV to about −40 mV, or about −20 mV, −25 mV, −30 mV, −35 mV, or −40 mV, where any value can be a lower and upper endpoint of a range (e.g., −30 mV to −305 mV). Not wishing to be bound by theory, the negative charge is due to the ionization and deprotonation of glutathione and the nitric oxide compound on the surface of the antibacterial nanoparticles.

The antibacterial nanoparticles described herein possess unique and desirable optical properties. The antibacterial nanoparticles have a broad absorption range in the visible region. In one aspect, the antibacterial nanoparticles absorb visible light in the range of from about 350 nm to about 550 nm, or about 350 nm, 375 nm, 400 nm, 425 nm, 450 nm, 475 nm, 500 nm, 425 nm, or 550 nm, where any value can be a lower and upper endpoint of a range (e.g., 375 nm to 525 nm). The antibacterial nanoparticles in the presence of light can generate more photons, resulting in more efficient therapeutic behavior. Under visible light exposure, the antibacterial nanoparticles can produce singlet oxygen (¹O₂), superoxide (O₂·—), hydroxyl (·HO), and (ONOO⁻) radicals. Therefore, during photodynamic activity, each radical plays a role in inducing oxidative stress and causing oxidative damage to targeted cells.

In addition to producing radicals useful in photodynamic therapy, the antibacterial nanoparticles described herein release nitric oxide that can be effective in the treatment or prevention of a bacterial infection. The antibacterial nanoparticles can provide a sustained release of nitric oxide. In one aspect, the antibacterial nanoparticle provides sustained release of nitric oxide in the amount of from about 100 mol min⁻¹ mg⁻¹ to about 300 mol min⁻¹ mg⁻¹ in an aqueous solution after one hour in the absence of light, or about 100 mol min⁻¹ mg⁻¹, 150 mol min⁻¹ mg⁻¹, 200 mol min⁻¹ mg⁻¹, 250 mol min⁻¹ mg⁻¹, or 300 mol min⁻¹ mg⁻¹, where any value can be a lower and upper endpoint of a range (e.g., 150 mol min⁻¹ mg⁻¹ to 250 mol min⁻¹ mg⁻¹). In another aspect, the antibacterial nanoparticles can provide sustained release of nitric oxide in the amount of from about 200 mol min⁻¹ mg 1 to about 500 mol min⁻¹ mg⁻¹ in an aqueous solution after one hour when exposed to light at an energy of from about 40 J/cm² to about 50 J/cm², or about 200 mol min⁻¹ mg⁻¹, 250 mol min⁻¹ mg⁻¹, 300 mol min⁻¹ mg⁻¹, 350 mol min⁻¹ mg⁻¹, 400 mol min⁻¹ mg⁻¹, 450 mol min⁻¹ mg⁻¹, or 500 mol min⁻¹ mg⁻¹, where any value can be a lower and upper endpoint of a range (e.g., 250 mol min⁻¹ mg⁻¹ to 400 mol min⁻¹ mg⁻¹). The antibacterial nanoparticles are stable and can be stored as solid or liquid compositions in the dark for extended periods of time.

The antibacterial nanoparticles described herein are biocompatible. As demonstrated in the Examples, the antibacterial nanoparticles are nontoxic to mammalian cells and tissues. The biocompatibility of the antibacterial nanoparticles makes them useful in numerous biomedical applications. Furthermore, the ability of the nanoparticles to produce high yields of singlet oxygen (¹O₂), hydroxyl radical (·HO), superoxide radical (O₂·), and peroxynitrite (ONOO⁻) free radicals as well as release nitric oxide upon exposure to light make the antibacterial nanoparticles effective in antimicrobial photodynamic therapy (APDT) against difficult strains of bacterial infection such as, for example, Gram-positive methicillin-resistant S. aureus (MRSA) and Gram-negative E. coli.

In one aspect, the antibacterial nanoparticles described herein can be used in wound healing. For example, the antibacterial nanoparticles can be incorporated into hydrogels, which can be subsequently used in wound dressings. In one aspect, the antibacterial nanoparticles can be mixed with one or more polymers and water to produce hydrogels. For example, the antibacterial nanoparticles can be mixed with alginate solution and crosslinked to make an alginate hydrogel containing the antibacterial nanoparticles. The hydrogel can include polymers such as, for example, alginate, gelatin, polyethylene glycol, polyvinyl alcohol, a poloxamer, or any combination thereof. The hydrogel can be an amorphous gel or can be incorporated into an article such as a wound dressing having an adhesive layer and/or a barrier material.

In another aspect, the antibacterial nanoparticles described herein can be included in a matrix material or scaffold. In one aspect, the matrix material can be a hydrogel, sponge, a natural or synthetic fiber (e.g., a suture), a polymeric film, nanofibers, etc. Polymeric solutions including solvents that do not dissolve the antibacterial nanoparticles described herein can be used to form a matrix. In one aspect, a solution of the antibacterial nanoparticles and a polymer such as, for example, polylactic acid can be electrospun or cast to form polymeric nanofibers or films containing the antibacterial nanoparticles described herein.

In one aspect, medical devices and/or medical grade polymer substrates can be impregnated with the antibacterial nanoparticles described herein. Medical devices of the present disclosure and/or medical grade polymer substrates can include, but are not limited to commercially available tubing (e.g. PharMed® BPT, PureFit® SBP, PureFit® SMP, PureFit® SVP, PureFit® SWP, SaniPure™ BDF™, SaniPure™ 60, Sani-Tech® LA-60, Sani-Tech® Sil-250, Sani-Tech® STHT™-C, Sani-Tech® STHT™-R, Sani-Tech® STHT™-R-HD, Sani-Tech® STHT™-WR, Sani-Tech® STHT™-W, CO, Tygon® 2275, Tygon® 2275 I.B., Tygon® 3350, Tygon® 3355L, Tygon® 3360LA, Tygon® 3370 I.B., Tygon® LFL, Tygon® Lab (R-3603), Tygon® LFL, Tygon® Food (B-44-4X), Tygon® Fuel & Lubricant (F-4040-A), Tygon® Chemical (2001), Versilic® SPX-50, Versilic® SPX-70 I.B., Silicone (platinum-cured), Silicone (peroxide-cured), BioPharm Silicone and BioPharm Plus Silicone (platinum-cured), Puri-Flex™, C-FLEX®, PharMed® BPT, PharmaPure®, GORE® STA-PURER PCS, GORE® STA-PURE® PFL, PTFE, Norprene® (A 60 G), Norprene® Food (A 60 F), Chem-Durance® Bio, GORE® Style 400, Viton®).

In one aspect, the medical grade polymer of the present disclosure, or medical device made therefrom, is treated (e.g., impregnated) with the antibacterial nanoparticles. The surface modifications of the present disclosure can be applied to a medical grade polymers on a wide variety of medical devices including various surfaces of such devices that are associated with the cause or production of infection once administered to the subject. For example, catheters such as urinary catheters, represent a common site of infection once administered to the subject, with approximately 95% of all hospital-acquired UTI's being associated with urinary catheters and 87% of hospital-acquired bloodstream infections associated with blood vessel catheters. Thus, the surface modifications of the present disclosure can be applied to medical devices such as catheters, including surfaces such as the interior lumen and/or and exterior of catheters. The compositions and methods of the present disclosure may also be used for various other medical device applications where fungal and/or bacterial infection is prevalent when the medical device is administered to the subject such as endotracheal tubes and extracorporeal membrane oxygenation.

Embodiments of the present disclosure include medical devices made of or comprising parts made of the medical grade polymer substrates of the present disclosure described above. Ideally, medical devices or parts that will be in sustained contact with a subject (e.g., those parts in sustained contact with a subject's tissues (e.g., skin, blood, epithelium, etc.) are made of the medical grade polymer substrates of the present invention. In one aspect, medical devices of the present disclosure include catheters (e.g, urinary catheters, blood vessel catheters, etc.) or other medical tubing (e.g., endotracheal tubing, nephorsomy tubing, colostomy tubing) or medical ports, and the like.

In other aspects, the antibacterial nanoparticles can be used in cancer treatment. As discussed above, under visible light irradiation the antibacterial nanoparticles generate high yields of singlet oxygen (¹O₂), hydroxyl radical (·HO), superoxide radical (O₂·), and peroxynitrite (ONOO⁻) free radicals that can enhance antimicrobial photodynamic therapy (APDT). The antibacterial nanoparticles are dynamic in their ability to specifically target pathogenic infections while remaining nontoxic towards mammalian cells and can also be used as medical device coatings to prevent infections, as well as in treatment and management of diseases like cancer and autoimmune skin disorders. The use of the antibacterial nanoparticles provides a non-invasive approach to the treatment of cancer when the antibacterial nanoparticles are administered topically to the subject and subsequently exposed to visible light.

In various aspects, the present disclosure relates to pharmaceutical compositions comprising a therapeutically effective amount of the antibacterial nanoparticles and a pharmaceutically-acceptable carrier. As used herein, “pharmaceutically-acceptable carrier” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of the antibacterial nanoparticles, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

In one aspect, the pharmaceutical compositions can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the antibacterial nanoparticles, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

### Aspects

Aspect 1. An antibacterial nanoparticle produced by the method comprising
admixing a porphyrin with glutathione or a pharmaceutically acceptable salt or ester thereof to produce a first compound; and
reacting the first compound with a nitric oxide compound, wherein the nitric oxide compound forms a covalent bond with glutathione or the pharmaceutically acceptable salt or ester thereof.

Aspect 2. The antibacterial nanoparticle of Aspect 1, wherein the porphyrin and glutathione or the pharmaceutically acceptable salt or ester thereof are admixed with a base.

Aspect 3. The antibacterial nanoparticle of Aspect 1, wherein the base comprises an alkali metal hydroxide or alkaline earth metal hydroxide.

Aspect 4. The antibacterial nanoparticle of any one of Aspects 1-3, wherein the porphyrin has the structure I

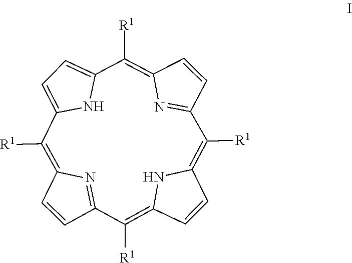

wherein R¹ are each a substituted or unsubstituted aryl group, a substituted or unsubstituted alkyl group, a substituted or unsubstituted cycloalkyl group, a carboxylic acid or ester, an amino group, or an azido group.

Aspect 5. The antibacterial nanoparticle of any one of Aspects 1-4, wherein the porphyrin further comprises a transition metal coordinated to the porphyrin.

Aspect 6. The antibacterial nanoparticle of any one of Aspects 1-5, wherein the porphyrin is 5,10,15,20-tetra(4-pyridyl)-21H,23H-porphine.

Aspect 7. The antibacterial nanoparticle of any one of Aspects 1-6, wherein the molar ratio of porphyrin to glutathione or the pharmaceutically acceptable salt or ester thereof is from about 0.5:1 to about 2:1.

Aspect 8. The antibacterial nanoparticle of any one of Aspects 1-6, wherein the molar ratio of porphyrin to glutathione or the pharmaceutically acceptable salt or ester thereof is about 1:1.

Aspect 9. The antibacterial nanoparticle of any one of Aspects 1-8, wherein the first compound is isolated prior to step (b).

Aspect 10. The antibacterial nanoparticle of any one of Aspects 1-9, wherein the nitric oxide compound is a S-nitrosothiol compound.

Aspect 11. The antibacterial nanoparticle of any one of Aspects 1-9, wherein the nitric oxide compound is nitrosated cysteine.

Aspect 12. An antibacterial nanoparticle comprising a porphyrin and a nitric oxide compound covalently bonded to glutathione or a pharmaceutically acceptable salt or ester thereof.

Aspect 13. The antibacterial nanoparticle of Aspect 12, wherein the nitric oxide compound is a S-nitrosothiol compound.

Aspect 14. The antibacterial nanoparticle of Aspect 13, wherein the S-nitrosothiol compound is S-nitroso-N-acetyl-penicillamine, S-nitroso-N-acetylcysteine, S-nitroso-N-acetyl cysteamine, S-nitrosoglutathione, S-nitrosocysteamine-glutathione, methyl S-nitrosothioglycolate, nitrosated cysteine, or any combination thereof.

Aspect 15. The antibacterial nanoparticle of Aspect 13 or 14, wherein the porphyrin has the structure I

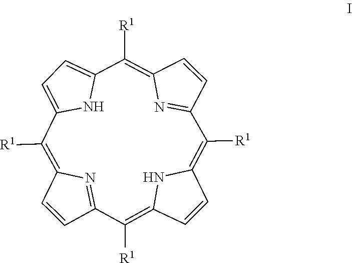

wherein R¹ are each a substituted or unsubstituted aryl group, a substituted or unsubstituted alkyl group, or a substituted or unsubstituted cycloalkyl group.

Aspect 16. The antibacterial nanoparticle of any one of Aspects 12-15, wherein the porphyrin further comprises a transition metal coordinated to the porphyrin.

Aspect 17. The antibacterial nanoparticle of any one of Aspects 12-16, wherein the porphyrin is 5, 10, 15,20-tetra(4-pyridyl)-21H,23H-porphine.

Aspect 18. The antibacterial nanoparticle of any one of Aspects 1-17, wherein the nanoparticle is biocompatible.

Aspect 19. The antibacterial nanoparticle of any one of Aspects 1-17, wherein the nanoparticle is an octahedral.

Aspect 20. The antibacterial nanoparticle of Aspect 19, wherein the octahedral has an edge length of from about 100 nm to about 120 nm.

Aspect 21. The antibacterial nanoparticle of any one of Aspects 1-20, wherein the nanoparticle has an average size of about 100 nm to about 200 nm.

Aspect 22. The antibacterial nanoparticle of any one of Aspects 1-20, wherein the nanoparticle has a polydispersity index from about 0.1 to about 0.3.

Aspect 23. The antibacterial nanoparticle of any one of Aspects 1-20, wherein the nanoparticle has a zeta potential of from about-20 mV to about-40 mV.

Aspect 24. The antibacterial nanoparticle of any one of Aspects 1-20, wherein the nanoparticle provides sustained release of nitric oxide in the amount of from about 100 mol min⁻¹ mg 1 to about 300 mol min⁻¹ mg⁻¹ in an aqueous solution after one hour in the absence of light.

Aspect 25. The antibacterial nanoparticle of any one of Aspects 1-20, wherein the nanoparticle provides sustained release of nitric oxide in the amount of from about 200 mol min⁻¹ mg⁻¹ to about 500 mol min⁻¹ mg⁻¹ in an aqueous solution after one hour when exposed to light at an energy of from about 40 J/cm² to about 50 J/cm².

Aspect 26. A pharmaceutical composition comprising the antibacterial nanoparticle of any one of Aspects 1-25 and a pharmaceutically acceptable carrier.

Aspect 27. A method for treating or preventing a bacterial infection in a subject in need thereof comprising administering to the subject the antibacterial nanoparticle of any one of Aspects 1-25.

Aspect 28. The method of Aspect 27, wherein the nanoparticles are exposed to visible light.

Aspect 29. The method of Aspect 27 or 28, wherein the nanoparticles kill Gram-positive MRSA and Gram-negative E. coli.

Aspect 30. An article comprising the antibacterial nanoparticle of any one of Aspects 1-25.

Aspect 31. The article of Aspect 30, wherein the article comprises a wound dressing or a suture.

Aspect 32. The article of Aspect 30, wherein the article comprises a medical device.

Aspect 33. The article of Aspect 32, wherein the medical device comprises a catheter or medical tubing comprising a urinary catheter, a blood vessel catheter, an endotracheal tubing, a nephrostomy tubing, a colostomy tubing, or a medical port.

Aspect 34. A method for treating cancer in a subject comprising administering to the subject the antibacterial nanoparticle of any one of Aspects 1-25.

Aspect 35. The method of Aspect 34, wherein the antibacterial nanoparticle is administered topically to the subject.

Aspect 36. The method of Aspect 34 or 35, wherein the antibacterial nanoparticle is exposed to visible light after the antibacterial nanoparticle is administered to the subject.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Materials and Methods

### Materials

All required chemicals were used without further purification unless otherwise noted. 5,10,15,20-Tetra(4-pyridyl)-21H,23H-porphine, sodium hydroxide, 1-Ethyl-3-[3-(dimethylamino)-propyl]carbodiimide hydrochloride, N-hydroxysuccinimide, cystine, cetrimonium bromide (CTAB), hydrochloric acid (HCl, 1 M), sodium hydroxide (NaOH, 1 M) and all other chemicals were purchased from Sigma-Aldrich (St. Louis, MO). and glutathione (GSH), were purchased from GOLD BIO (St. Louis, MO) Cell Counting Kit-8 (CCK-8) was purchased from Enzo Life Sciences (Farmingdale, NY). Phosphate-buffered saline (PBS, pH 7.4, containing 138 mM NaCl, 2.7 mM KCl, and 10 mM sodium phosphate) was used for all in vitro experiments. Dulbecco's modified Eagle's medium (DMEM) and trypsin-EDTA were obtained from Corning (Manassas, VA 20109). Fetal bovine serum (FBS) and penicillin-streptomycin (Pen-Strep) were bought from Gibco Life Technologies (Grand Island, NY). 3T3 mouse fibroblast cells (ATCC 1658) were originally obtained from American Type Culture Collection (ATCC) CRL-1658™. Singlet oxygen green sensor (SOSG), HAPF, APF, and Nitric oxide dye DMF-FM was purchased from Thermo Fisher Scientific (Suwanee, GA). 5-Cyano-2,3-Ditolyl Tetrazolium Chloride (CTC, Biotium, USA), All solutions were prepared in ultrapure water (resistivity of 18.2 MΩ·cm).

### Methods

### Synthesis of Porphyrin Nanoparticles (POP-NPs)

The porphyrin nanoparticles stabilized GSH, POP (GSH)-NPs were synthesized by following reported protocol with modification.³⁶ Briefly, 0.5 mL freshly prepared stock solution of 5,10,15,20-Tetra(4-pyridyl)-21H,23H-porphine (0.01 M) dissolved in 0.2 M HCl was added dropwise to 9.5 ml aqueous solution containing 0.01 M of GSH and NaOH (0.015 M) and the reaction was continuously stirred for 72 h at room temperature (25° C.). The purification of NPs was done through centrifugation (12000 rpm for 20 minutes) and washed 3 times with Mili-Q water to remove the free surfactants.

Further functionalization of S-nitrosocysteine (Cys-NO) with POP (GSH)-NPs was carried out according to the procedure of Kumar et al.⁷⁷ GSH is rich in functional groups (two-COOH groups and one —NH₂ group), providing the possibility of conjugating the NH₂ group of the Cys-(NO) and the carboxyl group of GSH onto the surface of the NPs (FIG. 1). Briefly, an aqueous solution of POP-GSH-NPs (6 mg) was resuspended in water and then aqueous solutions of N-(3-dimethylaminopropyl)-N-ethylcarbodiimide hydrochloride (EDC; 0.02 mM) and N-hydroxysuccinimide (NHS; 0.05 mM) were added to give a total volume of 5 ml to the reaction mixture. The reaction mixture was stirred for 30 min after which Cys-NO (0.00043 mmol) was added dropwise to the same reaction system which was stirred for an additional 24 h at 4° C. After completion of the reaction process, the POP-GSH/Cys-NO-NPs were centrifuged at 12,000 rpm for 20 min at 4° C. NPs were purified twice with Milli-Q water at 4° C. The NPs were lyophilized, and the yield of dried product was calculated to be 5.2 mg after recovery.

A typical method for the synthesis of CTAB stabilized POP-NPs was carried out according following a reported protocol.³⁶

### Characterization of Nanoparticles

The lyophilized, powders were resuspended in Milli-Q water and sonicated for 2-3 minutes in a water bath at room temperature. NPs were deposited on silicon substrates for SEM analysis and TEM grids to characterize the morphology of the NPs. The SEM images were taken using a Thermo Fisher Scientific (FEI) Teneo microscope. Transmission Electron Microscopy (TEM) analysis was performed using a FEI Tecnai20 with 200 kV acceleration voltage and equipped with a Gatan slow scan CCD camera. The physical characteristics of the NPs (i.e., size distribution and zeta-potential) were characterized using a dynamic light scattering (DLS) instrument (Malvern Zetasizer Nano S90). The UV-vis measurements were performed using a Cary 50 Conc UV-vis spectrometer. Infrared spectroscopy (FTIR) studies of the POP-NPs were carried out using a Thermo Nicolet Model 6700 spectrometer using a KBr pellet loading technique. Samples were analyzed in the wavelength region 4000-650 cm⁻¹ at 128 scans with a 4 cm⁻¹ resolution.

### Scanning Electron Microscopy Study of Bacterial Cells

Scanning electron microcopy (SEM, FEI Teneo, FEI Co.) was utilized to examine the surface morphology of the nanoparticles. All samples were sputter-coated with gold-palladium using a Leica sputter coater (Leica Microsystems), and an accelerating voltage of 5 kV was employed to examine the samples.

### Nitric Oxide Release Analysis

Nitric oxide (NO) release kinetics were measured using a Sievers Chemiluminescence 280i NO Analyzer (NOAs, GE Analytical, Boulder, Co). Samples were immersed in PBS buffer solution (pH=7.4) and kept in a dark reaction vessel maintained at 37° C. to simulate physiological conditions. To measure the amount of NO released from the nanoparticles over time, N₂ sweep gas was continuously bubbled into the reaction vessel at 200 mL min⁻¹ to purge any NO present in the buffer into the chemiluminescence detection chamber. When present in the chamber, NO reacts with ozone to generate nitrogen dioxide at an excited state (NO₂·). When returning to the ground state, photons emitted are detected to measure NO concentrations. Using a calibration constant (mol ppb⁻¹ s⁻¹) and the concentration of nanoparticles present, cumulative NO release and release rates were then calculated. To measure the light-triggered NO release from POP NPs, at different time points the samples were placed in clear sample vials and irradiated with LED white lamp (12 W). Between each measurement, the samples were incubated at 37° C. in dark. The power (W) of the light and exposure time is converted into total energy deposition (dose ˜46 J/cm²) and were keep constant throughout the experiment.

### Measurement of Reactive Oxygen Species

Mouse fibroblast (3T3) cells (5×10⁴ cells/mL) were seeded into a 96 well plate overnight and treated with 10-100 μg/ml of POP (GSH)-Cys-NO-NPs. Next, each fluoresce probe was added to a well at a final concentration of 15 μM for each assay and irradiated with or without light (dose, 46 J/cm²) for various time intervals. A microplate reader (Tecan Infinite M200 Reader) was used to acquire the fluorescence signals immediately before and after illumination from SOSG (Ex: 525, Em: 505 nm), HPF (Ex: 490, Em: 515), and DHE (Ex: 580, Em: 480 nm) fluorescence dyes. To further detect peroxynitrite, the fluorescence (Ex/Em=490/530 nm) was measured for the assay. Three separate experiments were run for each assay, using untreated samples as a control during the experiment.

### Cell Culture

Mouse fibroblast cells (NIH/3T3) were obtained from ATCC (Manassas, VA, USA). Cells were cultured in HyClone Dulbecco's Modified Eagle Medium DMEM (Gibco, Invitrogen, Carlsbad, CA, USA) growth medium supplemented with 10% fetal bovine serum (Gibco, Invitrogen, Carlsbad, CA, USA), 2 mM L-glutamine 100 U/mL penicillin (MediaTech, USA), and 100 μg/MI streptomycin (MediaTech). Cells were maintained in a humidified atmosphere containing 5% CO2, and temperature was maintained at 37° C.

### Measurement of Cell Cytotoxicity (CCK-8 Assay)

The cell viability of 3T3 cells was determined by CCK-8 method using a microplate reader (BioTek's Synergy Mx, USA). For this, around 3×10³ cells per well were seeded into a 96-well plate and allowed to culture overnight until the cells fully adhered to the bottom of the plate. The cells were treated with different treatment concentrations of POP-NPs for 72h in both dark and light conditions. Each treatment group had a final drug concentration 2-500 μg/ml of POP-NPs in the growth medium. The treated cells were incubated for 6 h and then irradiated with a light dose of 46 J/cm² (measured with a power meter) and further plates were incubated with respect to treatment conditions.

After that, CCK-8 reagent (10 μl, according to manufacture recommendation) was added to each well. The cells were further incubated for 4 h at 37° C. The medium from each well was then removed and transferred to a new 96 well plate. The plate was gently shaken for 3 min (instrument setup) to homogenize medium, and the absorbance at 420 nm was recorded by a microplate reader. Each experiment condition was run three times, and the data were shown as the mean value plus a standard deviation (+SD).

### Live and Dead Assay

The mouse fibroblast cells (2×10⁴ cells) were seeded into a chamber slide Nunc Lab Tek Chamber Slides (Thermo-Fisher Scientific, USA) and incubated with 100 μg/ml of the POP-NPs for 6 h and then irradiated with a white light dose of 46 J/cm² similar to cell cytotoxicity. Serum-free DMEM medium containing 500 μL of calcein AM (5 μM, ThermoFisher Scientific, USA) and EthD-III (Biotium, USA, Cat. #40050) (5 μM) was added to each well and allowed to further incubate for 30-35 min at 37° C. In viable cells, calcein-AM hydrolyzed by esterases in the cytoplasm releases the green, fluorescent dye, calcein. Meanwhile, EthD-III, a DNA dye that is impermeable to an intact plasma membrane, translocates the dead cells and binds to the nucleus DNA to give out red fluorescence. To remove the excess free dye molecules, the cells were washed with serum-free medium three times and imaged using a confocal laser microscope (Zeiss, LSM 710, USA) with the following setup. Calcein-AM Ex/Em=493 nm/520 nm; EthD-III Ex/Em=553 nm/568 nm.

### Intracellular Detection of ROS/NO Species

The intracellular NO release analysis was performed as reported by Kumar et al.⁷⁸ The mouse fibroblast cells (2×10⁴ cells) were seeded into Nunc Lab Tek Chamber Slides (Thermo-Fisher Scientific, USA), incubated with 100 μg/ml of the POP-NPs for 6 h, and then irradiated with a white light dose of 46 J/cm² similar to cell cytotoxicity. The NO released from POP (GHS)-Cys-NO-NPs would then react with DAF-FM (5 μM) to produce green fluorescence. Meanwhile, EthD-III, a DNA dye that is impermeable to an intact plasma membrane, translocates dead cells and binds to the nucleus DNA to give out red fluorescence. To remove free dye molecules, the cells were washed with serum-free medium three times and imaged under a confocal laser microscope (Zeiss, LSM 710, USA) with the following setup. DAF-FM, Ex/Em=495 nm/515 nm; EthD-III ex/em=553 nm/568 nm.

### Photodynamic Effects on Microorganism

Gram-negative E. coli (ATCC 25922) and Gram-positive methicillin-resistant S. aureus (MRSA; ATCC BAA 041) were cultured in LB broth at 37° C. on a shaker incubator at 150 rpm for 12 h. The bacterial cultures were washed with sterile PBS and re-suspended in sterile PBS at a concentration of ˜10⁸ CFU/ml (corresponding to 0.1 OD at 600 nm measured using UV-Vis spectrophotometer). In a 96-well plate, 100 UL of the bacterial solution was added to POP (GSH)-NPs and POP (GHS)-Cys-NO-NPs with different concentrations (n=4) and the cells were provided a light dose of 46 J/cm²; at the same time a plate without radiation was used in dark conditions for a comparison study. The plate was incubated for 24 h at 37° C. on a shaker incubator at 150 rpm. Post incubation, 100 μl from the wells were pipetted into a microcentrifuge tube containing 900 μl sterile PBS and serially diluted. 20 μl from the serially diluted solutions were plated on LB agar plates and incubated for 24 h. The colonies formed on the LB plates were counted and the reduction in viability of the bacteria was calculated according to the following equation (Eqn. 1).

$$\% Reduction in viability = \frac{{C}_{control} - {C}_{sample}}{{C}_{control}} \qquad ( Eqn . 1 )$$

### Where, C=CFU/ml

### Measurement of Bacterial Respiration

The microorganisms S. aureus (ATCC BAA 041) and E. coli (ATCC 25922) bacteria cells were cultured in LB Broth/Lennox (BioShop Lab Science Products) in an orbital incubator (37° C., 130 rpm), until the optical density reached 0.1, which corresponds to approximately 108 CFU per mL. To examine the respiratory activity, 100 μl of the bacterial solution was seeded into a 96 well plate and 100 μg/ml of POP (GSH)-NPs or POP (GSH)-Cys-NO-NPs was added and then incubated for 3 h in orbital shaker at 37° C. The 5-Cyano-2,3-Ditolyl Tetrazolium Chloride (CTC, Biotium, USA) was dissolved in biological grade pure water to prepare 50 mM stock solution, and the solution was slowly stirred overnight in the dark at room temperature. The freshly prepared stock solution (50 μM/per well) was used for each set of experiments. The cells were irradiated to deliver a light dose of 46 J/cm² and transferred to a microplate reader to record an absorbance at 450 nm/630 nm (reduced state). Cells treated in the dark were incubated covered with aluminum foil for the same time as the PDI groups for the result evaluation.

### Statistical Analysis

All values are expressed as average±SEM (standard error of the mean), which represents the standard deviation of the sample mean estimate of a population mean. Experiments were repeated at least three times with comparable results. Student's t-test where P<0.05 was statistically significant between two groups. Results were considered statistically significant with a confidence level of 95% (p<0.05).

### Results and Discussion

### Synthesis and Characterization of POP (GSH)-Cys-NO-NPs

GSH stabilized POP-NPs were synthesized to develop a more biocompatible, highly stable, and well-dispersed nanoparticle system. The NPs were assessed for their applicability towards ADPT therapy. POP-NPs have been previously used in materials science but have not been widely explored for biomedical applications. According to previous reports, most of the POP-NPs used for biomedical applications are synthesized using surfactants (e.g. CTAB, MTAB, SDS, SDL, and P123) that are toxic to mammalian cells.^{44,45} To eliminate these effects, different polymer functionalities were used for the surface modification of noncarriers that take additional synthesis and purifications steps.⁴⁶ Due to IT-IT stacking and rigid planar structures, porphyrin and its derivatives can naturally aggregate in aqueous conditions which results in fluorescence quenching and reduced ROS production efficiency due to an aggregation-caused quenching (ACQ) effect.⁴⁷ This subsequently reduces the therapeutic efficacy of the designed system.⁴⁸ Therefore, we took advantage of the lower molecular weight, more biocompatible, and nontoxic properties of GSH in the synthesis of POP-NPs.

POP (GSH)-Cys-NO-NPs were synthesized in a two-step process. POP-NPs were first prepared using a GSH-assisted interfacial self-assembly process. The self-assembly process was initiated by the noncovalent interaction such as hydrophilic and hydrophobic and IT-TT stacking between the individual porphyrin molecules (building block) and GSH (stabilizing agent) which initiate nucleation to form a well-defined nanostructure POP (GSH)-NPs. Further, nitrosated cysteine (Cys-NO) was functionalized with GSH available on the NPs surface through EDC/NHS coupling (FIG. 1). Depending on kinetic conditions and the surfactant used, results can vary in the formation of different shapes and sizes of the NPs.^{36, 49} Here we obtained smaller, nearly monodispersed NPs with octahedral geometries by replacing CTAB, a toxic surfactant that has been used previously to prepare porphyrin particles.^{36, 49}

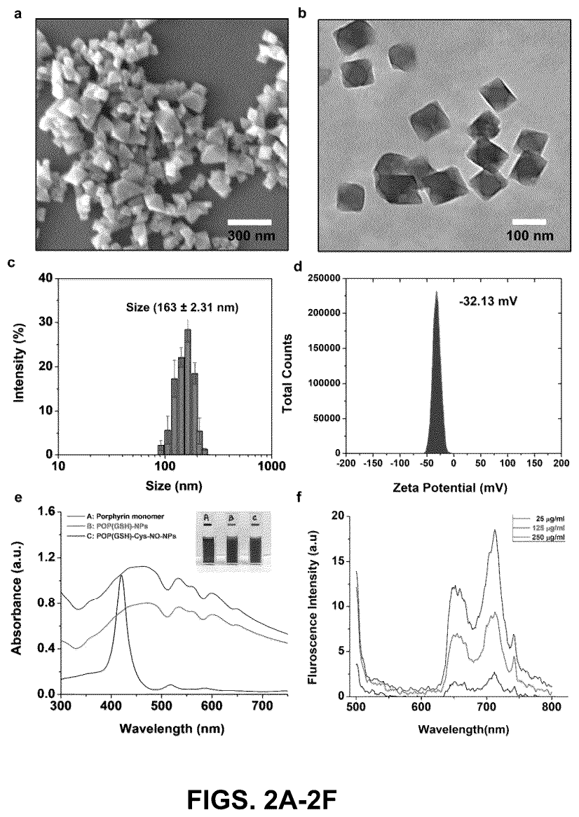

POP-NPs were characterized by scanning electron microscopy (SEM), transmission electron microscopy (TEM), UV-visible spectroscopy, fluorescence spectroscopy, and FTIR spectroscopy. The representative SEM images of the resultant POP (GSH)-Cys-NO-NPs show a well-defined external morphology with an average edge length of ˜143 nm with 2.3% standard deviation, octahedral geometry, and uniform size distribution (FIG. 2a). TEM analysis also clearly shows nontetrahedral NPs with a clear edge of 103±2.4 nm in length (clear lines visible in octahedral shape) and a well-defined 3D-octahedral shape, uniform dispersion, and show uniform electron contrast without apparent defects (FIG. 2b). The dynamic light scattering (DLS, FIG. 2c) also showed that the resulting POP (GSH)-Cys-NO-NPs has an average size of 163±2.31 nm. The polydispersity index (PDI) was found to be 0.18±0.01, suggesting narrow size distribution and good water dispersibility. The nanoparticle surface was negatively charged (zeta-potential-31.91 mV, FIG. 2d) due to ionization and deprotonation of GSH and cysteine on to the NPs surfaces, indicating the successful stabilization of NPs during the synthesis.

The optical properties of POP (GSH)-Cys-NO-NPs exhibit different characteristics than the parent molecule (monomer). The absorption spectra of the NPs show five intense band characteristics because of the properties of porphyrin (according to the Gouterman model).⁵⁰ The absorption spectra (FIG. 2e) indicate that the intense Soret band of the parent molecules at 424 nm split after self-assembly with a red shift band arising at 442 nm (FIG. 2e), which indicates J-aggregation during the formation of NPs.⁵¹ Meanwhile, the absorption spectra of the NPs show a broader range in the visible region compared to the parent molecule. The ‘B’ band of the NPs ranges from 375 to 545 nm, which is three times broader compared with the monomer range (390-444 nm). This indicates that in the presence of light, the POP (GSH)-Cys-NO-NPs can generate more photons, resulting in more efficient therapeutic behavior. Most of the photosensitizers show maximum absorption within the therapeutic window (ε_{˜}103 M⁻¹ cm⁻¹ at 638-646 nm). The longest wavelength (700 nm) absorption band is essential for cancer therapy as red light shows more efficient tumor tissue penetration. Additionally, the intense absorption at 420 nm can be useful for antimicrobial therapy because photons generated by a porphyrin photosensitizer at 420 nm is five times higher than the number of photons at 630 nm.⁵²

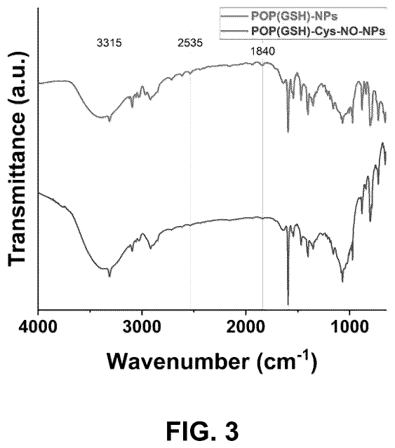

The stabilization of POP-NPs with GSH was confirmed by FTIR (FIG. 3) which identified characteristic GSH peaks at 3315, 2535, and 1840 cm⁻¹ that correspond to N—H stretching, S-H stretching, and C═O stretching vibrations, respectively.⁵³ Further functionalization of POP (GSH) NPs with EDC/NHS coupling to Cys-NO strengthened N—H stretching at 3315 cm⁻¹. Although thiol quantification from Cys-NO was inconclusive, corroborating results from NOA analysis show the successful conjugation of the NO donor to POP (GSH)-NPs.⁵⁴

To further evaluate the fluorescence properties, we recorded the fluorescence spectra of the NPs at different concentrations in an aqueous solution and found the fluorescence intensity increased as the concentration of the POP-NPs increased (FIG. 1f). The NPs did not show any fluorescence quenching, further confirming the stability of the NPs in an aqueous solution.

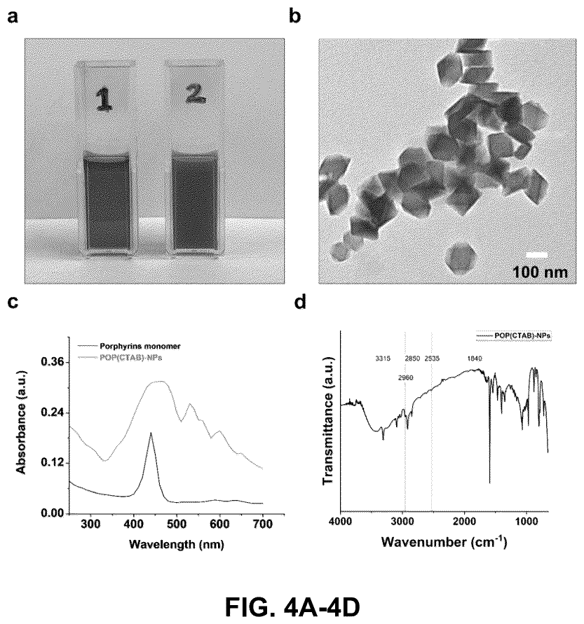

For the comparison study, POP-NPs stabilized by CTAB, POP (CTAB)-NPs were also synthesized and characterized through UV spectra, TEM, and IR spectra mentioned in detail in the supporting information (FIG. 4).

### Nitric Oxide Release Analysis

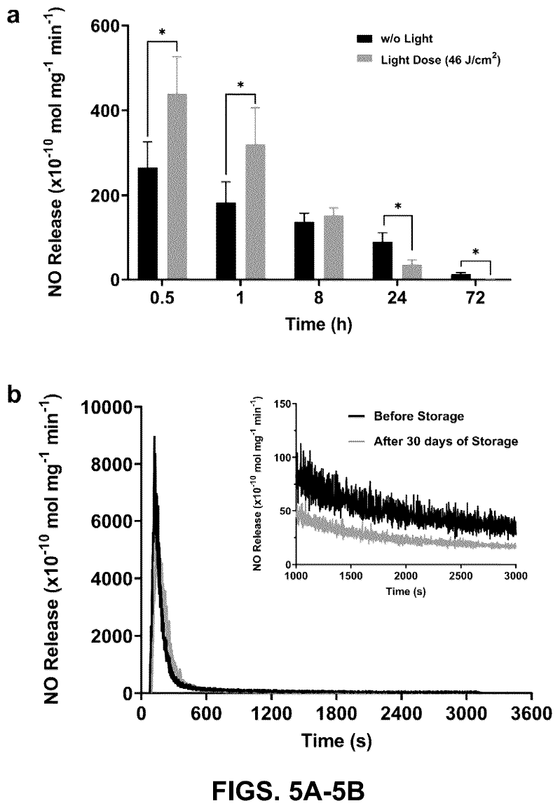

Due to the emergence of antibiotic resistance, developing alternative antimicrobial strategies to treat infections is crucial. The broad-spectrum antimicrobial activity of NO is well documented,⁵⁵ making NO-based nanoplatforms a promising tool for combatting infection. In this study, we recorded the NO release profiles of POP (GSH)-Cys-NO-NPs suspended in phosphate-buffered saline (PBS) with and without light exposure. As shown in FIG. 5a, the POP (GSH)-Cys-NO-NPs showed a sustained NO release rate of 183.7±48.6×10⁻¹⁰ mol min⁻¹ mg⁻¹ in dark conditions and 320.3±86.2×10⁻¹⁰ mol min⁻¹ mg⁻¹ in light conditions (46 J/cm²) after 1 h of immersion in PBS. The NO release kinetics were monitored and periodically measured during 72 h of incubation at 37° C. Interestingly, the initial average NO release rate of POP (GSH)-Cys-NO-NPs exposed to a 46 J/cm² light source (439.6±86.7×10⁻¹⁰ mol min⁻¹ mg⁻¹) was significantly higher than the average NO release rate of POP (GSH)-Cys-NO-NPs in dark conditions (264.7±61.6×10⁻¹⁰ mol min⁻¹ mg⁻¹) after 30 min of immersion in PBS (p<0.05). This can be expected as the catalytic release of NO from S-nitrosothiols (RSNOs) including S-nitrosocysteine can be achieved through visible light irradiation.⁵⁶ Conversely, after 24 h, the POP (GSH)-Cys-NO-NPs in dark conditions showed a significantly higher average NO release rate (90.3±21.3×10⁻¹⁰ mol min⁻¹ mg⁻¹) compared to in light conditions (35.5±11.4×10⁻¹⁰ mol min⁻¹ mg⁻¹), demonstrating that the NO supply stored within the nanoparticles had started to become exhausted. These results suggest promising tunable NO release from a POP (GSH)-Cys-NO-NPs. Moreover, similar NO delivery systems have been linked to decreased bacterial viability in previous literature.⁵⁷⁻⁶⁰ Therefore, to better understand the effects of the different NO delivery rates, the cytotoxicity and antimicrobial properties of the nanoparticles are later studied.

To assess the stability of the POP (GSH)-Cys-NO-NPs, the nanoparticles were similarly evaluated after one month of storage at −20° C. (FIG. 5b). After one month, the POP (GSH)-Cys-NO-NPs released a cumulative total of 14943.0±2491.1×10⁻¹⁰ mol mg⁻¹ after 60 min of immersion, while freshly prepared samples released a cumulative total of 11361.5±2844.2×10⁻¹⁰ mol mg⁻¹ after 60 min of immersion. There was no significant difference between the cumulative total NO release before or after an extended storage period (p>0.05), confirming that the POP (GSH)-Cys-NO-NPs are a durable, reliable system for NO delivery potentially capable of combatting infection.

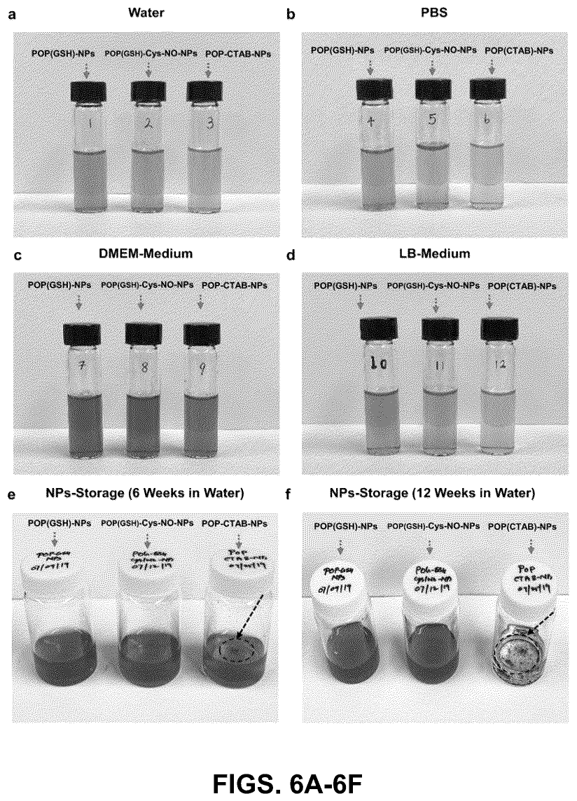

The POP (GSH)-Cys-NO-NPs were found to be stable in water and physiological conditions including phosphate buffer (pH=7.4) and cell culture medium under the influence of electrostatic interaction of the surface charge of the NPs (FIG. 6a-6d). POP (GSH)-Cys-NO-NPs were found to be very stable during storage for 3 months at 4° C. in dark conditions compared with CTAB stabilized NPs. No aggregation or precipitation was observed when the NPs were stored in an aqueous solution. However, CTAB stabilized NPs aggregated and became a clear solution during 3 months of storage (FIG. 6e, 6f).

### Photosensitizer Properties of POP (GSH)-Cys-NO-NPs (Formation of Radicals)

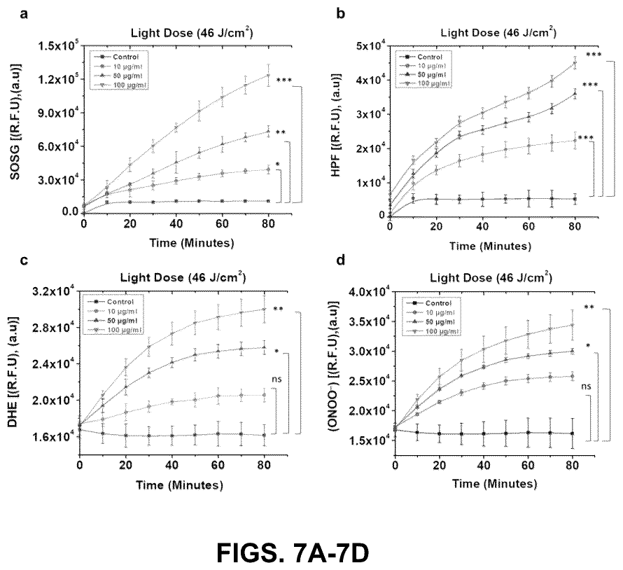

Considering the potential applications of POP (GSH)-Cys-NO-NPs for APDT, their abilities to generate reactive oxygen (ROS) and nitrogen species (RNS) were evaluated using specific ROS probes in the presence of visible light irradiation at a dose of 46 J/cm², as well as in cell proliferating conditions. Cell proliferating conditions were selected to mimic the in vivo application of designed NPs in the treatment of bacterial infections and diseases. Each of the fluorescence probes, sensitive to the specific radicals, were used to confirm ROS and RNS generation. The Singlet Oxygen Sensor Green (SOSG) is mainly sensitive to ¹O₂, the hydroxyphenyl fluorescein (HPF) is more sensitive to hydroxyl radicals (·HO), and dihydroethidium (DHE) is a redox-sensitive probe used to detect superoxide radicals (O₂·—). Under visible light exposure, it was found that there was significant production of singlet oxygen (102), superoxide (O₂·—), hydroxyl (·HO), and the generation of peroxynitrite (ONOO⁻) radicals were detected as compared to untreated control (FIG. 7). At a concentration of 100 μg/ml and under visible light exposure (46 J/cm²) POP (GSH)-Cys-NO-NPs showed a 10.2-fold increase in single oxygen species (¹O₂) FIG. 7a, 7.73-fold increase in hydroxyl radicals (·OH) FIG. 7b, 1.27-fold increase in superoxide (O₂·—) FIG. 7c, and 1.59-fold increase in the generation of peroxynitrite (ONOO⁻) compared to control FIG. 7d. These fluorescence probes indicate the relative contribution of the different ROS due to the photodynamic effect of the POP-(GSH)-Cys-NO-NPs.⁶¹ Furthermore, the NO release from the NPs could react with superoxide (OH·) during irradiation to form peroxynitrite, which is highly reactive due to the presence of unpaired valence shell electrons or non-static bonds that destroys cellular constituent efficiently and kills bacterial cells more effectively.⁶² Therefore, during photodynamic activity each radical plays a role in inducing oxidative stress and causing oxidative damage to targeted cells.⁶²

### Cell Toxicity and Biocompatibility of Nanoparticles

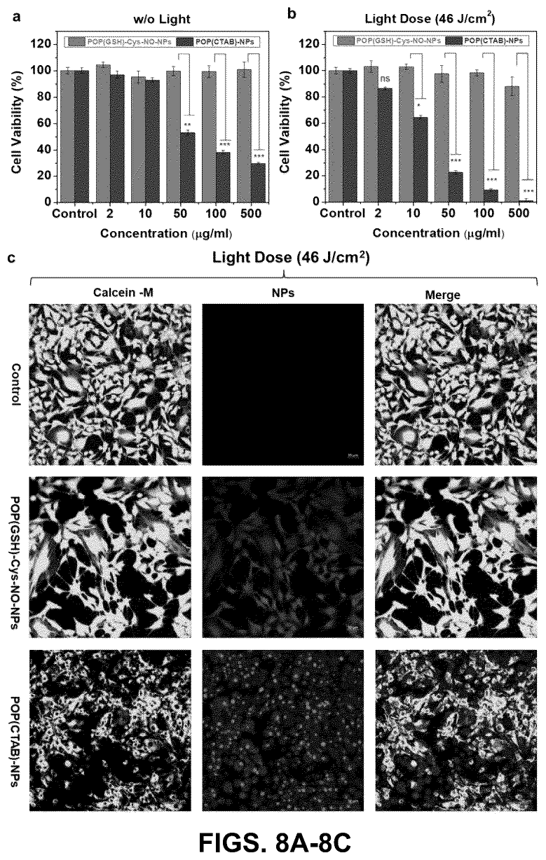

Recently several photosensitizer-based nanomaterials and their applications have been investigated in biomedical science. However, these nanomaterials still face several challenges during their clinical journey, and ultimately most of the materials do not graduate to the clinical stage due to the failure in biocompatibility (i.e. unacceptable toxicity).⁶³ Therefore, to evaluate the biocompatibility of the materials, we compared the toxicity of the nanoparticles using a Cell Counting Kit-8 (CCK-8) assay as well as a lived/dead fluorescence-based assay to investigate the effects of POP (GSH)-Cys-NO-NPs and POP (CTAB)-NPs on normal mouse fibroblast cells. The cells were treated for 72 h with POP-NPs in concentrations ranging from 2 to 500 μg/ml in both light and dark conditions. Control experiments were run separately with similar conditions without dye labeling to evaluate this result. The results show that in both conditions POP (GSH)-Cys-NO-NPs were highly biocompatible (FIG. 8a, b) and did not cause any toxicity towards the cells. On the other hand, a significant reduction in cell viability was observed with POP (CTAB)-NPs. For POP (CTAB)-NPs under dark conditions, 100 and 500 μg/ml treatments had 61.56±1.65% and 70.58±0.86% reduction in viability relative to POP (GSH)-Cys-NO-NPs at the same respective dosages. Under light condition, cell viability upon exposure to POP-(GSH)-Cys-NO-NPs was 90.57±1.02% and 98.87±1.12% relative to POP (CTAB)-NPs (FIG. 8a, b). These results show that the POP (GSH)-Cys-NO-NPs are nontoxic at equivalent concentrations and light exposures in comparison with CTAB stabilized POP-NPs.

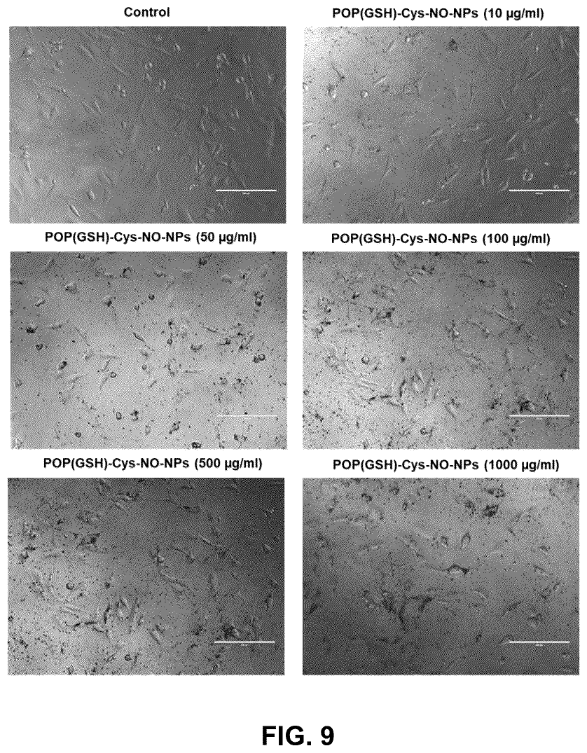

To further confirm this result, we observed the morphology of the cells under similar treatment conditions using confocal microscopy and a live/dead cell assay. The confocal microscopy results also show no sign of toxicity in the case of POP (GSH)-Cys-NO-NPs (FIG. 8c). The majority of cells retained viability after internalization of NPs within the cells (red channel). Calcein is a non-fluorescent, cell membrane-impermeable dye that permeates the intact cell membrane where the acetoxymethyl (AM) group is cleaved by intracellular esterases, thus yielding green fluorescence in the cytoplasm of viable cells with intact plasma membranes.⁶⁴ Ethidium homodimer (EthD-III) enters cells and binds to nucleic acids, resulting in bright red fluorescence in the nucleus of the dead cells. Fluorescence was detected in the cell cytoplasm of the POP (GSH)-Cys-NO-NPs (red channel) due to the fluorescent properties of the NPs, but no sign of toxicity (red signal in the nucleus) was observed, which parallelly confirmed by running a separate control experiment with NPs. Bright-field microscopy images also show that cells looked healthy and there was no change in the cell's morphology observed after treatment with POP (GSH)-Cys-NO-NPs (FIG. 9).

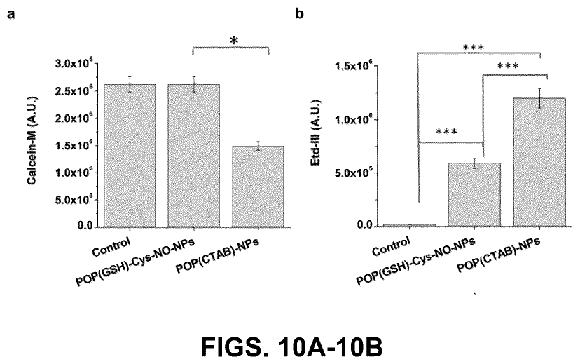

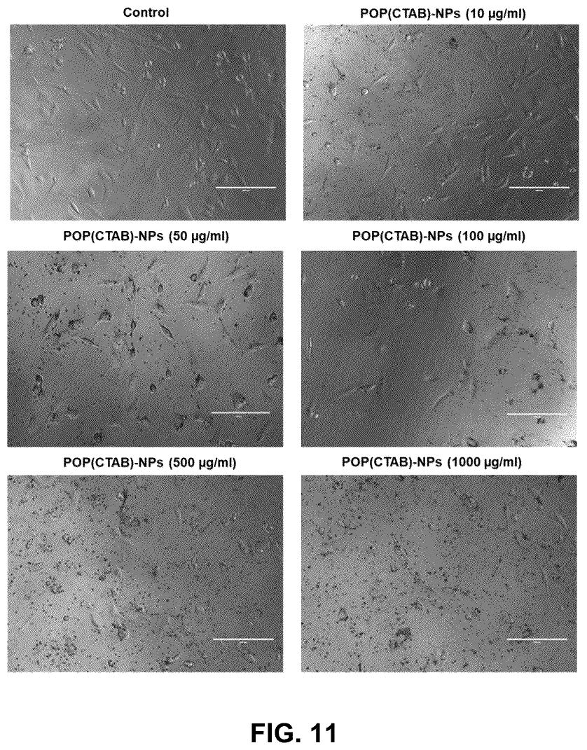

However, a significant change in the cell's integrity was observed in the case of CTAB stabilized, POP (CTAB)-NPs. Dead cells either did not stain with calcein due to lack of esterase activity or failure to retain calcein (decreased by 1.52-fold relative to unirradiated cells, Figure S4a) in the cytoplasm due to loss of plasma membrane integrity as a result of material toxicity. After loss of viability, EthD-III penetrates the compromised membranes of dead cells efficiently and stains the nucleus with brighter red fluorescence (1.66-fold increase relative to unirradiated cells, FIG. 10),⁶⁴ indicating that the POP (CTAB)-NPs caused significant reduction in cell viability and toxicity to the normal cells which was also observed very clearly under bright field after treatment with POP-CTAB-NPs (FIG. 11).

### Intracellular Release of NO and its Biocompatibility

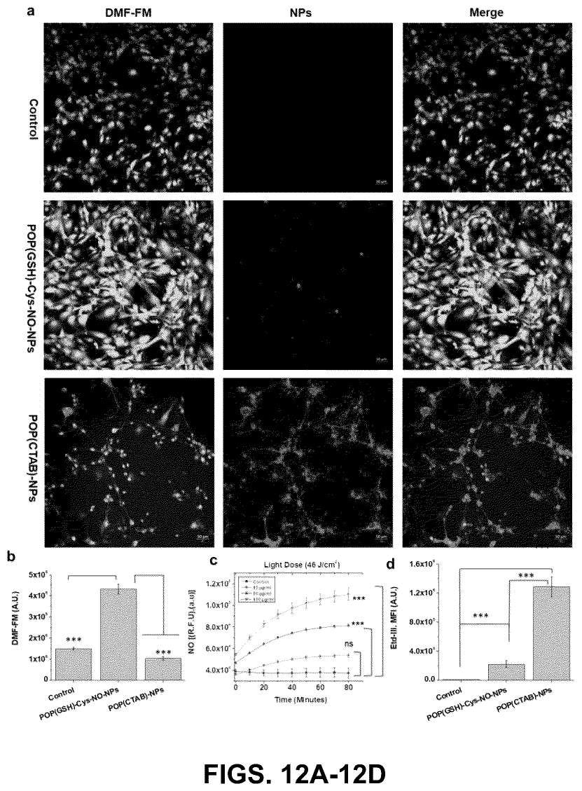

We further examined the intracellular localization of NO and its influence on cellular morphology and the level of toxicity towards normal cells. The release of NO in a cellular system was labeled by a NO labeling dye (DAM-FM, fluorometric sensor of NO) and Eth-Ill dye used as a marker for DNA damage. The cells were incubated with POP (GSH)-Cys-NO and POP (CTAB)-NPs at a similar concentration of 100 μg/ml for 24 h with the same light dose used in the cellular cytotoxicity studies. In this study, NO slowly releases from POP (GSH)-Cys-NO-NPs and internalizes within the cells, which results in emission of the green fluorescence (FIG. 12a, b). As compared to control significant increment of green fluorescence (1.86-fold) was observed with NO functionalized POP-NPs with no sign of cytotoxicity. Some cell nuclei occasionally emitted brighter red fluorescence signals that might be due to overgrowth. Further we confirmed the release of NO through spectrophotometry analysis and found that a significant increase (˜1.97 fold) in emission of green fluorescence was observed in comparison to untreated controls (FIG. 12c), which confirms that NO release from the POP-NPs was internalized within the cells. Exposure to POP (CTAB)-NPs resulted in a dramatic, observable alteration in cell morphology and toxicity towards the cells. The cells lost membrane integrity, and a simultaneous increase in red fluorescence (95.56-fold) was observed throughout the cells due to DNA damage compared with POP (GSH)-Cys-NO-NPs-NPs and untreated controls (FIG. 12a, d). These results indicate that the designed POP-NPs functionalized with NO were found to be highly biocompatible and did not cause cellular damage to the normal cells. Therefore, further studies evaluated the antimicrobial photodynamic therapy of POP (GSH)-Cys-NO-NPs.

### Photodynamic Effects of POP (GSH)-Cys-NO-NPs

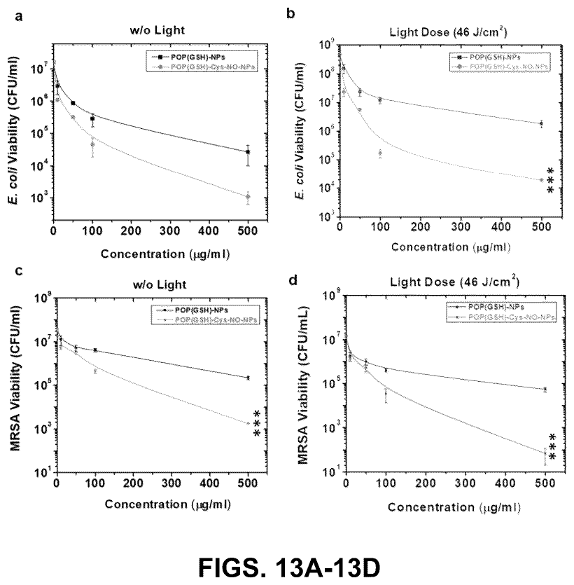

The antibacterial photodynamic effects of POP (GSH)-NPs and POP (GSH)-Cys-NO-NPs were evaluated against Gram-positive MRSA and Gram-negative E. coli with varying (10, 50, 100, and 500 μg/ml) of NPs in presence of light. The colony counting method was employed to quantify reduction efficiency and log reduction of bacterial viability in the presence of the NPs (with or without light). MRSA infections have become endemic in hospital settings and have become widespread over the years 65. Gram-negative E. coli has long been studied as a model bacterium for various antimicrobial susceptibility testing and infections, 66 but both strains have the propensityto form biofilms and be a menace in a biomedical context.^{67, 68} Table 1 represents the reduction efficiency of the NPs with varying concentrations in the presence and absence of visible light. It was observed that with increasing concentrations of both POP (GSH)-NPs and POP (GSH)-Cys-NO-NPs, the antibacterial efficacy increases. Secondly, POP (GSH)-Cys-NO-NPs show enhanced antibacterial activity at all the concentrations when compared to POP (GSH)-NPs as shown in FIG. 13. It is expected, as POP (GSH)-Cys-NO-NPs generate NO in the presence of heat, moisture, and light as stimulants.⁶⁹ It is a well-established fact that NO demonstrates efficient antibacterial effect through the formation of reactive nitrogen species (RNS) and through nitrosative and oxidative action.⁷⁰

TABLE 1
Comparative analysis of reduction in the viability of bacterial CFU/mL in the presence of POP(GSH) and POP(GSH)-
Cys-NO-NPs in light and dark conditions. Comparisons are relative to control untreated POP-NPs.

| Treatment condition | | | POP(GSH)-NPs | | | | POP(GSH)-Cys-NO-NPs | | | |
|---|---|---|---|---|---|---|---|---|---|---|
| Concentration (μg/ml) | | | 10 | 50 | 100 | 500 | 10 | 50 | 100 | 500 |
| E. coli | Dark | Log reduction | 0.73 | 1.25 | 1.74 | 2.77 | 1.16 | 1.69 | 2.54 | 4.17 |
| | | Reduction | 81.36 ± | 94.41 ± | 98.19 ± | 99.83 ± | 93.08 ± | 97.97 ± | 99.71 ± | 99.96 ± |
| | | efficiency (%) | 10.58 | 0.88 | 0.96 | 0.11 | 0.63 | 0.25 | 0.20 | 0.01 |
| | Light | Log reduction | 0.48 | 1.29 | 1.58 | 2.40 | 1.30 | 1.91 | 3.43 | 4.38 |
| | | Reduction | 66.82 ± | 94.93 ± | 97.39 ± | 99.60 ± | 95.02 ± | 98.78 ± | 99.96 ± | 99.99 ± |
| | | efficiency (%) | 12.06 | 1.84 | 0.79 | 0.13 | 1.52 | 0.12 | 0.01 | 0.0003 |
| | p- values | (Dark vs. light) | 0.19 | 0.68 | 0.32 | 0.07 | 0.1 | 0.5 | 0.09 | 0.0076 |
| MRSA | Dark | Log reduction | 0.48 | 0.88 | 0.97 | 2.24 | 0.86 | 1.03 | 1.94 | 4.32 |
| | | Reduction | 67.25 ± | 86.69 ± | 89.3 ± | 99.42 ± | 86.06 ± | 90.56 ± | 98.85 ± | 99.99 ± |
| | | efficiency (%) | 15.63 | 4.75 | 2.71 | 0.13 | 2.63 | 2.96 | 0.27 | .001 |
| | Light | Log reduction | 0.92 | 1.16 | 1.56 | 2.42 | 0.98 | 1.45 | 2.62 | 5.32 |
| | | Reduction | 87.86 ± | 93.10 ± | 97.21 ± | 99.62 ± | 89.52 ± | 96.43 ± | 99.76 ± | 99.9995 ± |
| | | efficiency (%) | 5.00 | 2.18 | 0.68 | 0.10 | 1.79 | 1.23 | 0.18 | 0.000 |
| | p- values | (Dark vs. light) | 0.0952 | 0.1009 | 0.008 | 0.025 | 0.13 | 0.033 | 0.008 | 0.0001 |

However, when compared between light and dark conditions while incubating the NPs with bacterial cells, a statistically significant difference in the reduction efficiency was observed at higher concentrations for both bacterial strains. There was no significant difference between the reduction efficiency in the case of E. coli in the presence of POP (GSH)-NPs, in both light and dark conditions. For POP (GSH)-Cys-NO-NPs, the viability of bacterial cells had a statistically significant difference only at the 500 μg/ml concentration. The effect of the NPs on MRSA was more pronounced than on E. coli. At concentrations of 100 and 500 μg/ml, both POP (GSH)-NPs and POP (GSH)-Cys-NO-NPs had a significant difference in the viability of bacterial cells under dark and light conditions (Table 1). The microbial inactivation/death due to the photodynamic effect appears to be applicable at concentrations around 100-500 μg/ml of POP (GSH)-Cys-NO-NPs.

Reactive oxygen species (ROS) are formed in the presence of light which can attack a diverse range of targets in the bacterial cells. The presence of a photosensitizer is an integral part of photodynamic therapy involving the production of ROS in visible light. The two most important ROS that can damage bacterial cells are ·OH and ¹O₂.⁷¹ Due to the short half-life of NO as well as the reactive species, bacteria would not easily develop resistance against such an antimicrobial strategy. The combination of both NO and light-dependent generation of singlet oxygen, hydrogen peroxides, and peroxynitrites therefore improves the antibacterial efficiency categorically with increasing concentrations. From the results, POP (GSH)-Cys-NO-NPs are most suitable in the context of eliciting superior antibacterial activity while not compromising the viability of mammalian cells that can come into contact during therapeutic applications.

### Disruption of Bacterial Cell Metabolism

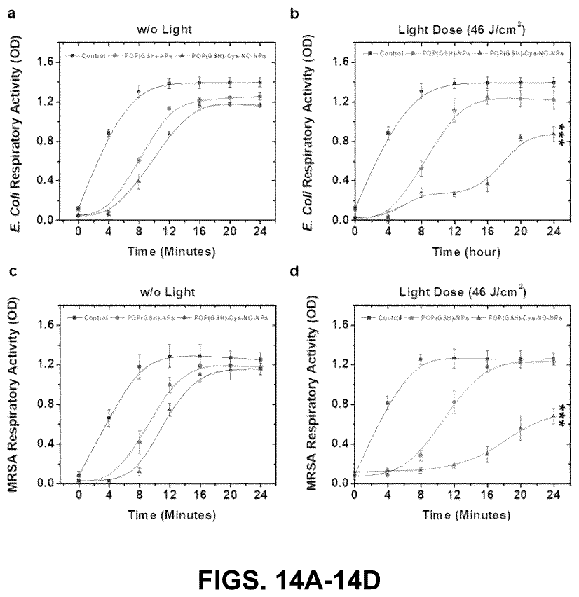

To further distinguish healthy versus unhealthy bacterial cell populations during treatment with POP-NPs, the respiratory activity of the bacterial cells was tested. 5-Cyano-2,3-ditolyl tetrazolium chloride (CTC) dye used in this study evaluates the electron transport chain in cellular respiration and has been used to evaluate the respiratory activity of many bacterial populations as well as populations undergoing drug efficacy testing.⁷² In the electron transport system, tetrazolium salts function as artificial redox partners instead of the final electron acceptor, oxygen.⁷³ Respiring or healthy bacteria take up the CTC and reduce it to insoluble formazan (CTC formazan), which accumulates in the cells. On the other hand, dead or inactive bacteria show no accumulation of CTC formazan because the dye competes with the terminal electron acceptor and eventually destroys the membrane potential of the cells once the reduction processes are completed.⁷⁴ During our study we found that when the POP-NPs were exposed to bacterial cells in presence of light (dose 46 J/cm²), bacterial respiration was reduced. Similarly, as the concentration of the NPs increased during the treatment, bacterial respiration decreased more in presence of light (FIG. 14). Even in the dark, slower rates of respiration were observed due to the therapeutic behavior of the NPs. Therefore, we conclude that after treatment with POP (GSH)-Cys-NO-NPs, cells stop respiring or respire at slower rates, which confirms that light exposure combined with NP treatment enhances the production of radicals which interact with the bacterial cell membranes and slows down/stops the respiration activity of the bacteria due to the photodynamic properties of the POP (GSH)-Cys-NO-NPs.

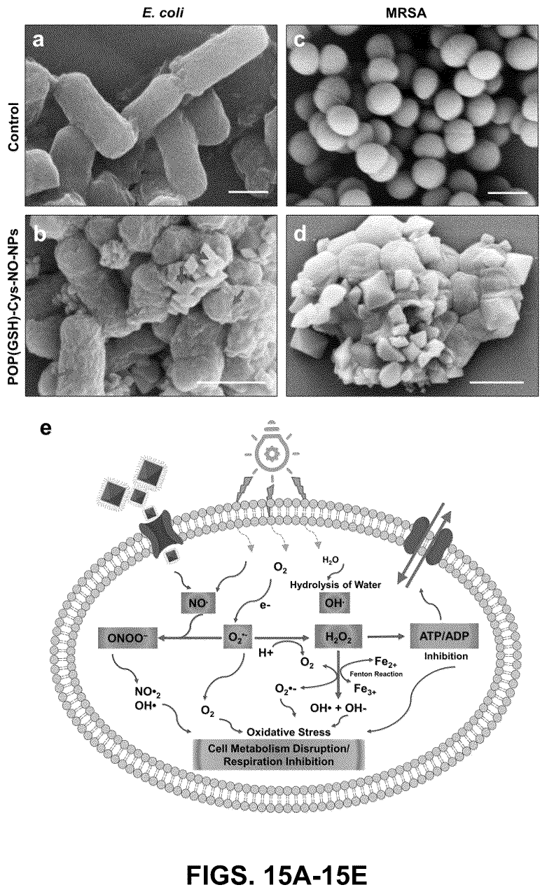

Finally, the effects of photodynamic behaviors of the POP-GSH-Cys-NO-NPs in altering the morphology of bacterial cells were investigated using SEM. Prior to light exposure, we found that the bacteria cells exhibited smooth, intact surfaces with well-defined morphologies (spherical and rod-shaped for MRSA and E. coli cells, respectively) and sizes typical for normal cells. However, after treatment with NPs with light irradiation, significant damage to their cellular structure was observed including deformed membranes and a loss in volume consistent with cell membrane leakage. MRSA and E. coli, ordinarily spherical and rod-shaped (respectively) with smooth, intact membranes, showed membrane disruption and increased surface roughness after exposure to NPs under light irradiation (FIG. 15a-d). Similar changes in cell morphology and membrane deformation have been observed in previous studies, indicating cell lysis and death.^{75, 76} Therefore, we conclude that POP (GSH)-Cys-NO attacked bacteria cells through formation of ROS and NO, leading to disruption of cell membrane, and inhibition of the electron transport chain, which leads to cellular death due to alteration of the bacterial respiration (metabolism) process FIG. 15e.

### CONCLUSION

GSH stabilized and Cys-NO functionalized, self-assembled porphyrin nanoparticle capable of enhancing the antimicrobial photodynamic activity for enhanced inhibition of Gram-positive MRSA and Gram-negative E. coli bacteria under visible light conditions were synthesized. The photodynamic activity of POP (GSH)-Cys-NO-NPs significantly enhanced due to dual strategy of intracellular ROS generation and NO functionalization. POP (GSH)-Cys-NO-NPs showed greater biocompatibility (nontoxic in nature), higher stability in ambient conditions (dark), and can be well dispersed in aqueous solution and physiological buffer in comparison with CTAB stabilized, POP (CTAB)-NPs. The developed NPs not only demonstrate excellent photosensitizer activity, but also exhibit NO delivery in a cellular system, resulting in a better therapeutic approach in the treatment and management of bacterial infections. In addition, POP (GSH)-Cys-NO-NPs eliminate bacterial cells through the formation of ROS and NO, leading to cell membrane damage and inhibition of the electron transport chain ultimately resulting in cell death by alteration of the bacterial respiration process.

The established APDT potential of POP-NPs as photosensitizers coupled with the antibacterial properties of NO through the assembled POP-GSH/Cys-NO-NPs may prove to be beneficial for treatment and management of infectious diseases. This still underexplored multimodal therapy is not based on “conventional” drugs, but entirely induced and controlled by light stimuli which can be used for a wide range of applications in biomedical science.

It should be emphasized that the above-described embodiments of the present disclosure are merely possible examples of implementations and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### REFERENCES

1. Bloom, D. E.; Cadarette, D., Infectious Disease Threats in the Twenty-First Century: Strengthening the Global Response. Frontiers in immunology 2019, 10, 549-549.
2. Fair, R. J.; Tor, Y., Antibiotics and bacterial resistance in the 21st century. Perspectives in medicinal chemistry 2014, 6, 25-64.
3. Aslam, B.; Wang, W.; Arshad, M. I.; Khurshid, M.; Muzammil, S.; Rasool, M. H.; Nisar, M. A.; Alvi, R. F.; Aslam, M. A.; Qamar, M. U.; Salamat, M. K. F.; Baloch, Z., Antibiotic resistance: a rundown of a global crisis. Infection and drug resistance 2018, 11, 1645-1658.
4. Andrade, F.; Rafael, D.; Videira, M.; Ferreira, D.; Sosnik, A.; Sarmento, B., Nanotechnology and pulmonary delivery to overcome resistance in infectious diseases. Advanced Drug Delivery Reviews 2013, 65 (13), 1816-1827.
5. Kaittanis, C.; Santra, S.; Perez, J. M., Emerging nanotechnology-based strategies for the identification of microbial pathogenesis. Advanced drug delivery reviews 2010, 62 (4-5), 408-423.
6. Baptista, P. V.; McCusker, M. P.; Carvalho, A.; Ferreira, D. A.; Mohan, N. M.; Martins, M.; Fernandes, A. R., Nano-Strategies to Fight Multidrug Resistant Bacteria-“A Battle of the Titans”. Frontiers in microbiology 2018, 9, 1441-1441.
7. Kumar, A.; Chen, F.; Mozhi, A.; Zhang, X.; Zhao, Y.; Xue, X.; Hao, Y.; Zhang, X.; Wang, P. C.; Liang, X.-J., Innovative pharmaceutical development based on unique properties of nanoscale delivery formulation. Nanoscale 2013, 5 (18), 8307-8325.
8. Kumar, A.; Zhang, X.; Liang, X.-J., Gold nanoparticles: Emerging paradigm for targeted drug delivery system. Biotechnology Advances 2013, 31 (5), 593-606.
9. Li, L.-L.; Wang, H., Infection-targeted bactericidal nanoparticles. Nature Biomedical Engineering 2018, 2 (2), 56-57.
10. Wiegand, C.; Abel, M.; Ruth, P.; Elsner, P.; Hipler, U. C., pH Influence on Antibacterial Efficacy of Common Antiseptic Substances. Skin Pharmacology and Physiology 2015, 28 (3), 147-158.
11. Bartek, I. L.; Reichlen, M. J.; Honaker, R. W.; Leistikow, R. L.; Clambey, E. T.; Scobey, M. S.; Hinds, A. B.; Born, S. E.; Covey, C. R.; Schurr, M. J.; Lenaerts, A. J.; Voskuil, M. I., Antibiotic Bactericidal Activity Is Countered by Maintaining pH Homeostasis in Mycobacterium smegmatis. mSphere 2016, 1 (4), e00176-16.
12. Wang, L.; Hu, C.; Shao, L., The antimicrobial activity of nanoparticles: present situation and prospects for the future. International journal of nanomedicine 2017, 12, 1227-1249.
13. Arsiwala, A. M.; Raval, A. J.; Patravale, V. B., Nanocoatings on implantable medical devices. Pharmaceutical Patent Analyst 2013, 2 (4), 499-512.
14. Liu, Y.; Qin, R.; Zaat, S. A. J.; Breukink, E.; Heger, M., Antibacterial photodynamic therapy: overview of a promising approach to fight antibiotic-resistant bacterial infections. Journal of clinical and translational research 2015, 1 (3), 140-167.
15. Cieplik, F.; Deng, D.; Crielaard, W.; Buchalla, W.; Hellwig, E.; Al-Ahmad, A.; Maisch, T., Antimicrobial photodynamic therapy-what we know and what we don't. Critical Reviews in Microbiology 2018, 44 (5), 571-589.
16. Abrahamse, H.; Hamblin, M. R., New photosensitizers for photodynamic therapy. The Biochemical journal 2016, 473 (4), 347-364.
17. Ghorbani, J.; Rahban, D.; Aghamiri, S.; Teymouri, A.; Bahador, A., Photosensitizers in antibacterial photodynamic therapy: an overview. Laser therapy 2018, 27 (4), 293-302.
18. Agostinis, P.; Berg, K.; Cengel, K. A.; Foster, T. H.; Girotti, A. W.; Gollnick, S. O.; Hahn, S. M.; Hamblin, M. R.; Juzeniene, A.; Kessel, D.; Korbelik, M.; Moan, J.; Mroz, P.; Nowis, D.; Piette, J.; Wilson, B. C.; Golab, J., Photodynamic therapy of cancer: An update. CA: A Cancer Journal for Clinicians 2011, 61 (4), 250-281.
19. Bhattacharyya, A.; Chattopadhyay, R.; Mitra, S.; Crowe, S. E., Oxidative stress: an essential factor in the pathogenesis of gastrointestinal mucosal diseases. Physiological reviews 2014, 94 (2), 329-354.
20. Dharmaraja, A. T., Role of Reactive Oxygen Species (ROS) in Therapeutics and Drug Resistance in Cancer and Bacteria. Journal of Medicinal Chemistry 2017, 60 (8), 3221-3240.
21. Slavin, Y. N.; Asnis, J.; Hafeli, U. O.; Bach, H., Metal nanoparticles: understanding the mechanisms behind antibacterial activity. Journal of Nanobiotechnology 2017, 15 (1), 65.
22. Dai, T.; Huang, Y.-Y.; Hamblin, M. R., Photodynamic therapy for localized infections—state of the art. Photodiagnosis Photodyn Ther 2009, 6 (3-4), 170-188.
23. Ucuncu, M.; Mills, B.; Duncan, S.; Staderini, M.; Dhaliwal, K.; Bradley, M., Polymyxin-based photosensitizer for the potent and selective killing of Gram-negative bacteria. Chemical Communications 2020.
24. Sabella, S.; Carney, R. P.; Brunetti, V.; Malvindi, M. A.; Al-Juffali, N.; Vecchio, G.; Janes, S. M.; Bakr, O. M.; Cingolani, R.; Stellacci, F.; Pompa, P. P., A general mechanism for intracellular toxicity of metal-containing nanoparticles. Nanoscale 2014, 6 (12), 7052-7061.
25. Sukhanova, A.; Bozrova, S.; Sokolov, P.; Berestovoy, M.; Karaulov, A.; Nabiev, I., Dependence of Nanoparticle Toxicity on Their Physical and Chemical Properties. Nanoscale research letters 2018, 13 (1), 44-44.
26. Ventola, C. L., Progress in Nanomedicine: Approved and Investigational Nanodrugs. P & T: a peer-reviewed journal for formulary management 2017, 42 (12), 742-755.
27. Jin, W.; Liang, G.; Zhong, Y.; Yuan, Y.; Jian, Z.; Wu, Z.; Zhang, W., The Influence of CTAB-Capped Seeds and Their Aging Time on the Morphologies of Silver Nanoparticles. Nanoscale research letters 2019, 14 (1), 81-81.
28. Cheng, W.; Dong, S.; Wang, E., Synthesis and Self-Assembly of Cetyltrimethylammonium Bromide-Capped Gold Nanoparticles. Langmuir 2003, 19 (22), 9434-9439.
29. Pratsinis, A.; Hervella, P.; Leroux, J.-C.; Pratsinis, S. E.; Sotiriou, G. A., Toxicity of Silver Nanoparticles in Macrophages. Small 2013, 9 (15), 2576-2584.
30. Ray, P. C.; Yu, H.; Fu, P. P., Toxicity and environmental risks of nanomaterials: challenges and future needs. J Environ Sci Health C Environ Carcinog Ecotoxicol Rev 2009, 27 (1), 1-35.
31. Amos-Tautua, B. M.; Songca, S. P.; Oluwafemi, O. S., Application of Porphyrins in Antibacterial Photodynamic Therapy. Molecules (Basel, Switzerland) 2019, 24 (13), 2456.
32. MESQUITA, M. Q.; DIAS, C. J.; GAMELAS, S.; FARDILHA, M.; NEVES, M. G. P. M. S.; FAUSTINO, M. A. F., An insight on the role of photosensitizer nanocarriers for Photodynamic Therapy. Anais da Academia Brasileira de Ciências 2018, 90, 1101-1130.
33. Khan, R.; Özkan, M.; Khaligh, A.; Tuncel, D., Water-dispersible glycosylated poly(2,5′-thienylene) porphyrin-based nanoparticles for antibacterial photodynamic therapy. Photochemical & Photobiological Sciences 2019, 18 (5), 1147-1155.
34. Gong, X.; Milic, T.; Xu, C.; Batteas, J. D.; Drain, C. M., Preparation and Characterization of Porphyrin Nanoparticles. Journal of the American Chemical Society 2002, 124 (48), 14290-14291.
35. Malatesti, N.; Munitic, I.; Jurak, I., Porphyrin-based cationic amphiphilic photosensitisers as potential anticancer, antimicrobial and immunosuppressive agents. Biophysical reviews 2017, 9 (2), 149-168.
36. Wang, J.; Zhong, Y.; Wang, L.; Zhang, N.; Cao, R.; Bian, K.; Alarid, L.; Haddad, R. E.; Bai, F.; Fan, H., Morphology-Controlled Synthesis and Metalation of Porphyrin Nanoparticles with Enhanced Photocatalytic Performance. Nano Letters 2016, 16 (10), 6523-6528.
37. Zhang, Y.; Newton, B.; Lewis, E.; Fu, P. P.; Kafoury, R.; Ray, P. C.; Yu, H., Cytotoxicity of organic surface coating agents used for nanoparticles synthesis and stability. Toxicology in vitro: an international journal published in association with BIBRA 2015, 29 (4), 762-768.
38. Schairer, D. O.; Chouake, J. S.; Nosanchuk, J. D.; Friedman, A. J., The potential of nitric oxide releasing therapies as antimicrobial agents. Virulence 2012, 3 (3), 271-279.
39. Vaughn, M. W.; Kuo, L.; Liao, J. C., Estimation of nitric oxide production and reaction rates in tissue by use of a mathematical model. Am J Physiol-Heart C 1998, 274 (6), H2163-H2176.
40. Kumar, A.; Radzievski, R.; Xie, J. In Free Radical: Nitric Oxide in Cancer Therapy, 2018.
41. Juedes, M. J.; Wogan, G. N., Peroxynitrite-induced mutation spectra of pSP189 following replication in bacteria and in human cells. Mutation Research/Fundamental and Molecular Mechanisms of Mutagenesis 1996, 349 (1), 51-61.
42. Hall, C. N.; Garthwaite, J., What is the real physiological NO concentration in vivo? Nitric Oxide 2009, 21 (2), 92-103.
43. Jones, M. L.; Ganopolsky, J. G.; Labbé, A.; Wahl, C.; Prakash, S., Antimicrobial properties of nitric oxide and its application in antimicrobial formulations and medical devices. Applied Microbiology and Biotechnology 2010, 88 (2), 401-407.
44. Inácio, Â. S.; Mesquita, K. A.; Baptista, M.; Ramalho-Santos, J.; Vaz, W. L. C.; Vieira, O. V., In vitro surfactant structure-toxicity relationships: implications for surfactant use in sexually transmitted infection prophylaxis and contraception. PloS one 2011, 6 (5), e19850-e19850.
45. Inácio, Â. S.; Mesquita, K. A.; Baptista, M.; Ramalho-Santos, J.; Vaz, W. L. C.; Vieira, O. V., In Vitro Surfactant Structure-Toxicity Relationships: Implications for Surfactant Use in Sexually Transmitted Infection Prophylaxis and Contraception. PLoS ONE 2011, 6 (5), e19850.
46. Heinz, H.; Pramanik, C.; Heinz, O.; Ding, Y.; Mishra, R. K.; Marchon, D.; Flatt, R. J.; Estrela-Lopis, I.; Llop, J.; Moya, S.; Ziolo, R. F., Nanoparticle decoration with surfactants: Molecular interactions, assembly, and applications. Surface Science Reports 2017, 72 (1), 1-58.
47. Zhen, S.; Wang, S.; Li, S.; Luo, W.; Gao, M.; Ng, L. G.; Goh, C. C.; Qin, A.; Zhao, Z.; Liu, B.; Tang, B. Z., Efficient Red/Near-Infrared Fluorophores Based on Benzo[1,2-b: 4,5-b′]dithiophene 1,1,5,5-Tetraoxide for Targeted Photodynamic Therapy and In Vivo Two-Photon Fluorescence Bioimaging. Advanced Functional Materials 2018, 28 (13), 1706945.
48. Guo, B.; Cai, X.; Xu, S.; Fateminia, S. M. A.; Liu, J.; Liang, J.; Feng, G.; Wu, W.; Liu, B., Decoration of porphyrin with tetraphenylethene: converting a fluorophore with aggregation-caused quenching to aggregation-induced emission enhancement. Journal of Materials Chemistry B 2016, 4 (27), 4690-4695.
49. Bai, F.; Sun, Z.; Wu, H.; Haddad, R. E.; Coker, E. N.; Huang, J. Y.; Rodriguez, M. A.; Fan, H., Porous One-Dimensional Nanostructures through Confined Cooperative Self-Assembly. Nano Letters 2011, 11 (12), 5196-5200.
50. Dąbrowski, J. M.; Pucelik, B.; Regiel-Futyra, A.; Brindell, M.; Mazuryk, O.; Kyzioł, A.; Stochel, G.; Macyk, W.; Arnaut, L. G., Engineering of relevant photodynamic processes through structural modifications of metallotetrapyrrolic photosensitizers. Coordination Chemistry Reviews 2016, 325, 67-101.
51. Keller, N.; Calik, M.; Sharapa, D.; Soni, H. R.; Zehetmaier, P. M.; Rager, S.; Auras, F.; Jakowetz, A. C.; Görling, A.; Clark, T.; Bein, T., Enforcing Extended Porphyrin J-Aggregate Stacking in Covalent Organic Frameworks. Journal of the American Chemical Society 2018, 140 (48), 16544-16552.
52. Pucelik, B.; Paczyński, R.; Dubin, G.; Pereira, M. M.; Arnaut, L. G.; Dąbrowski, J. M., Properties of halogenated and sulfonated porphyrins relevant for the selection of photosensitizers in anticancer and antimicrobial therapies. PLOS ONE 2017, 12 (10), e0185984.
53. Basu, S.; Panigrahi, S.; Praharaj, S.; Ghosh, S. K.; Pande, S.; Jana, S.; Pal, T., Dipole-dipole plasmon interactions in self-assembly of gold organosol induced by glutathione. New Journal of Chemistry 2006, 30 (9), 1333-1339.
54. Grossi, L.; Montevecchi, P. C., S-nitrosocysteine and cystine from reaction of cysteine with nitrous acid. A kinetic investigation. J Org Chem 2002, 67 (24), 8625-30.
55. Kutty, S. K.; Ka Kit Ho, K.; Kumar, N., Chapter 7-Nitric Oxide Donors as Antimicrobial Agents. In Nitric Oxide Donors, Seabra, A. B., Ed. Academic Press: 2017; pp 169-189.
56. Frost, M. C.; Meyerhoff, M. E., Synthesis, characterization, and controlled nitric oxide release from S-nitrosothiol-derivatized fumed silica polymer filler particles. J Biomed Mater Res A 2005, 72 (4), 409-19.
57. Hetrick, E. M.; Shin, J. H.; Stasko, N. A.; Johnson, C. B.; Wespe, D. A.; Holmuhamedov, E.; Schoenfisch, M. H., Bactericidal efficacy of nitric oxide-releasing silica nanoparticles. ACS Nano 2008, 2 (2), 235-46.
58. Hibbard, H. A. J.; Reynolds, M. M., Synthesis of novel nitroreductase enzyme-activated nitric oxide prodrugs to site-specifically kill bacteria. Bioorg Chem 2019, 93, 103318.
59. Jin, H.; Yang, L.; Ahonen, M. J. R.; Schoenfisch, M. H., Nitric Oxide-Releasing Cyclodextrins. J Am Chem Soc 2018, 140 (43), 14178-14184.
60. Reighard, K. P.; Schoenfisch, M. H., Antibacterial Action of Nitric Oxide-Releasing Chitosan Oligosaccharides against Pseudomonas aeruginosa under Aerobic and Anaerobic Conditions. Antimicrob Agents Chemother 2015, 59 (10), 6506-13.
61. Price, M.; Reiners, J. J.; Santiago, A. M.; Kessel, D., Monitoring Singlet Oxygen and Hydroxyl Radical Formation with Fluorescent Probes During Photodynamic Therapy. Photochemistry and Photobiology 2009, 85 (5), 1177-1181.
62. Phaniendra, A.; Jestadi, D. B.; Periyasamy, L., Free Radicals: Properties, Sources, Targets, and Their Implication in Various Diseases. Indian Journal of Clinical Biochemistry 2015, 30 (1), 11-26.
63. Naahidi, S.; Jafari, M.; Edalat, F.; Raymond, K.; Khademhosseini, A.; Chen, P., Biocompatibility of engineered nanoparticles for drug delivery. Journal of Controlled Release 2013, 166 (2), 182-194.
64. Decherchi, P.; Cochard, P.; Gauthier, P., Dual staining assessment of Schwann cell viability within whole peripheral nerves using calcein-AM and ethidium homodimer. Journal of Neuroscience Methods 1997, 71 (2), 205-213.
65. Nasim, A.; Thompson, M. M.; Naylor, A. R.; Bell, P. R. F.; London, N. J. M., The impact of MRSA on vascular surgery. Eur J Vasc Endovasc 2001, 22 (3), 211-214.
66. Mondal, A.; Douglass, M.; Hopkins, S. P.; Singha, P.; Tran, M.; Handa, H.; Brisbois, E. J., Multifunctional S-Nitroso-N-acetylpenicillamine-Incorporated Medical-Grade Polymer with Selenium Interface for Biomedical Applications. ACS Applied Materials & Interfaces 2019, 11 (38), 34652-34662.
67. Costerton, J. W.; Montanaro, L.; Arciola, C. R., Biofilm in implant infections: its production and regulation. Int J Artif Organs 2005, 28 (11), 1062-8.
68. Donlan, R. M.; Costerton, J. W., Biofilms: survival mechanisms of clinically relevant microorganisms. Clin Microbiol Rev 2002, 15 (2), 167-93.
69. Kim, J. O.; Noh, J. K.; Thapa, R. K.; Hasan, N.; Choi, M.; Kim, J. H.; Lee, J. H.; Ku, S. K.; Yoo, J. W., Nitric oxide-releasing chitosan film for enhanced antibacterial and in vivo wound-healing efficacy. Int J Biol Macromol 2015, 79, 217-25.
70. Carpenter, A. W.; Schoenfisch, M. H., Nitric oxide release: part II. Therapeutic applications. Chem Soc Rev 2012, 41 (10), 3742-52.
71. Vatansever, F.; de Melo, W. C.; Avci, P.; Vecchio, D.; Sadasivam, M.; Gupta, A.; Chandran, R.; Karimi, M.; Parizotto, N. A.; Yin, R.; Tegos, G. P.; Hamblin, M. R., Antimicrobial strategies centered around reactive oxygen species—bactericidal antibiotics, photodynamic therapy, and beyond. FEMS Microbiol Rev 2013, 37 (6), 955-89.
72. Kobayashi, T.; Mito, T.; Watanabe, N.; Suzuki, T.; Shiraishi, A.; Ohashi, Y., Use of 5-Cyano-2,3-Ditolyl-Tetrazolium Chloride Staining as an Indicator of Biocidal Activity in a Rapid Assay for Anti-Acanthamoeba Agents. 2012, 50 (5), 1606-1612.
73. Créach, V.; Baudoux, A.-C.; Bertru, G.; Rouzic, B. L., Direct estimate of active bacteria: CTC use and limitations. 2003, 52 (1), 19-28.
74. Smith, J. J.; McFeters, G. A., Mechanisms of INT (2-(4-iodophenyl)-3-(4-nitrophenyl)-5-phenyl tetrazolium chloride), and CTC (5-cyano-2,3-ditolyl tetrazolium chloride) reduction in Escherichia coli K-12. Journal of Microbiological Methods 1997, 29 (3), 161-175.
75. Pathak, S.; Chauhan, V. S., Rationale-based, de novo design of dehydrophenylalanine-containing antibiotic peptides and systematic modification in sequence for enhanced potency. Antimicrob Agents Chemother 2011, 55 (5), 2178-88.
76. Prince, A.; Sandhu, P.; Ror, P.; Dash, E.; Sharma, S.; Arakha, M.; Jha, S.; Akhter, Y.; Saleem, M., Lipid-II Independent Antimicrobial Mechanism of Nisin Depends On Its Crowding And Degree Of Oligomerization. Sci Rep 2016, 6, 37908.
77. Kumar, A.; Huo, S.; Zhang, X.; Liu, J.; Tan, A.; Li, S.; Jin, S.; Xue, X.; Zhao, Y.; Ji, T.; Han, L.; Liu, H.; Zhang, X.; Zhang, J.; Zou, G.; Wang, T.; Tang, S.; Liang, X.-J., Neuropilin-1-Targeted Gold Nanoparticles Enhance Therapeutic Efficacy of Platinum (IV) Drug for Prostate Cancer Treatment. 2014, 8 (5), 4205-4220.
78. Gao, S.; Zhang, W.; Wang, R.; Hopkins, S. P.; Spagnoli, J. C.; Racin, M.; Bai, L.; Li, L.; Jiang, W.; Yang, X.; Lee, C.; Nagata, K; Howerth, E. W.; Handa, H.; Xie, J.; Ma, Q.; Kumar, A., Nanoparticles Encapsulating Nitrosylated Maytansine To Enhance Radiation Therapy. ACS Nano 2020, 14 (2), 1468-1481.

1. An antibacterial nanoparticle produced by the method comprising
(a) admixing a porphyrin with glutathione or a pharmaceutically acceptable salt or ester thereof to produce a first compound; and
(b) reacting the first compound with a nitric oxide compound, wherein the nitric oxide compound forms a covalent bond with glutathione or the pharmaceutically acceptable salt or ester thereof.

2. The antibacterial nanoparticle of claim 1, wherein the porphyrin and glutathione or the pharmaceutically acceptable salt or ester thereof are admixed with a base.

3. The antibacterial nanoparticle of claim 1, wherein the base comprises an alkali metal hydroxide or alkaline earth metal hydroxide.

4. The antibacterial nanoparticle of claim 1, wherein the porphyrin has the structure I 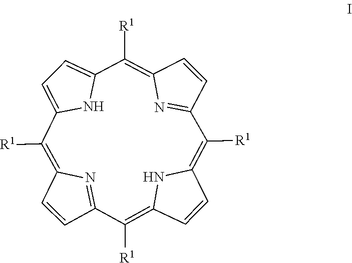
wherein R¹ are each a substituted or unsubstituted aryl group, a substituted or unsubstituted alkyl group, a substituted or unsubstituted cycloalkyl group, a carboxylic acid or ester, an amino group, or an azido group.

5. The antibacterial nanoparticle of claim 1, wherein the porphyrin further comprises a transition metal coordinated to the porphyrin.

6. The antibacterial nanoparticle of claim 1, wherein the porphyrin is 5,10,15,20-tetra(4-pyridyl)-21H,23H-porphine.

7. The antibacterial nanoparticle of claim 1, wherein the molar ratio of porphyrin to glutathione or the pharmaceutically acceptable salt or ester thereof is from about 0.5:1 to about 2:1.

8. The antibacterial nanoparticle of claim 1, wherein the molar ratio of porphyrin to glutathione or the pharmaceutically acceptable salt or ester thereof is about 1:1.

9. The antibacterial nanoparticle of claim 1, wherein the first compound is isolated prior to step (b).

10. The antibacterial nanoparticle of claim 1, wherein the nitric oxide compound is a S-nitrosothiol compound.

11. The antibacterial nanoparticle of claim 10, wherein the S-nitrosothiol compound is S-nitroso-N-acetyl-penicillamine, S-nitroso-N-acetylcysteine, S-nitroso-N-acetyl cysteamine, S-nitrosoglutathione, S-nitrosocysteamine-glutathione, methyl S-nitrosothioglycolate, nitrosated cysteine, or any combination thereof.

12. The antibacterial nanoparticle of claim 1, wherein the nitric oxide compound is nitrosated cysteine.

13. An antibacterial nanoparticle comprising a porphyrin and a nitric oxide compound covalently bonded to glutathione or a pharmaceutically acceptable salt or ester thereof.

14. The antibacterial nanoparticle of claim 13, wherein the nitric oxide compound is a S-nitrosothiol compound.

15. The antibacterial nanoparticle of claim 14, wherein the S-nitrosothiol compound is S-nitroso-N-acetyl-penicillamine, S-nitroso-N-acetylcysteine, S-nitroso-N-acetyl cysteamine, S-nitrosoglutathione, S-nitrosocysteamine-glutathione, methyl S-nitrosothioglycolate, nitrosated cysteine, or any combination thereof.

16. The antibacterial nanoparticle of claim 13, wherein the porphyrin has the structure I 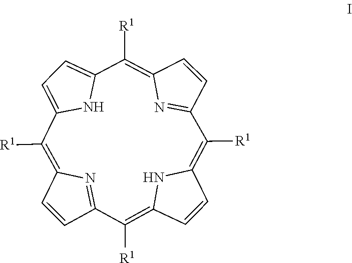
wherein R¹ are each a substituted or unsubstituted aryl group, a substituted or unsubstituted alkyl group, or a substituted or unsubstituted cycloalkyl group.

17. The antibacterial nanoparticle of claim 13, wherein the porphyrin further comprises a transition metal coordinated to the porphyrin.

18. The antibacterial nanoparticle of claim 13, wherein the porphyrin is 5,10,15,20-tetra(4-pyridyl)-21H,23H-porphine.

19. The antibacterial nanoparticle of claim 1, wherein the nanoparticle is biocompatible.

20. The antibacterial nanoparticle of claim 1, wherein the nanoparticle is an octahedral.

21. The antibacterial nanoparticle of claim 20, wherein the octahedral has an edge length of from about 100 nm to about 120 nm.

22. The antibacterial nanoparticle of claim 1, wherein the nanoparticle has an average size of about 100 nm to about 200 nm.

23. The antibacterial nanoparticle of claim 1, wherein the nanoparticle has a polydispersity index from about 0.1 to about 0.3.

24. The antibacterial nanoparticle of claim 1, wherein the nanoparticle has a zeta potential of from about −20 mV to about −40 mV.

25. The antibacterial nanoparticle of claim 1, wherein the nanoparticle provides sustained release of nitric oxide in the amount of from about 100 mol min⁻¹ mg⁻¹ to about 300 mol min⁻¹ mg⁻¹ in an aqueous solution after one hour in the absence of light.

26. The antibacterial nanoparticle of claim 1, wherein the nanoparticle provides sustained release of nitric oxide in the amount of from about 200 mol min⁻¹ mg⁻¹ to about 500 mol min⁻¹ mg⁻¹ in an aqueous solution after one hour when exposed to light at an energy of from about 40 J/cm² to about 50 J/cm².

27. A pharmaceutical composition comprising the antibacterial nanoparticle of claim 1 and a pharmaceutically acceptable carrier.

28. A method for treating or preventing a bacterial infection in a subject in need thereof comprising administering to the subject the antibacterial nanoparticle of claim 1.

29. The method of claim 28, wherein the nanoparticles are exposed to visible light.

30. The method of claim 28, wherein the nanoparticles kill Gram-positive MRSA and Gram-negative E. coli.

31. An article comprising the antibacterial nanoparticle of any one of claims 1-18.

32. The article of claim 31, wherein the article comprises a wound dressing or a suture.

33. The article of claim 31, wherein the article comprises a medical device.

34. The article of claim 33, wherein the medical device comprises a catheter or medical tubing comprising a urinary catheter, a blood vessel catheter, an endotracheal tubing, a nephrostomy tubing, a colostomy tubing, or a medical port.

35. A method for treating cancer in a subject comprising administering to the subject the antibacterial nanoparticle of claim 1.

36. The method of claim 35, wherein the antibacterial nanoparticle is administered topically to the subject.

37. The method of claim 35, wherein the antibacterial nanoparticle is exposed to visible light after the antibacterial nanoparticle is administered to the subject.
